(19) United States
(12) Patent Application Publication
(10) Pub. No.: US 2025/0368655 A1
(43) Pub. Date: Dec. 4, 2025

(54) SMALL MOLECULE INHIBITORS OF BCR-ABL

(71) Applicant: Oregon Health & Science University, Portland, OR (US)

(72) Inventors: Mallesh Pandrala, Portland, OR (US); Sanjay Malhotra, Portland, OR (US); Jeffrey W. Tyner, Portland, OR (US); Brian J. Druker, Portland, OR (US)

(73) Assignee: Oregon Health & Science University, Portland, OR (US)

(21) Appl. No.: 19/108,121

(22) PCT Filed: Aug. 29, 2023
(86) PCT No.: PCT/US2023/031451

§ 371 Date: Feb. 28, 2025

Related U.S. Application Data
(60) Provisional application No. 63/373,842, filed on Aug. 29, 2022.

(51) Int. Cl. C07D 487/04; A61K 31/5025; A61K 45/06; A61P 35/02

(52) U.S. Cl. CPC C07D 487/04; A61K 31/5025; A61K 45/06; A61P 35/02

(57) ABSTRACT

Provided are compounds of Formula (I) wherein X is selected from the group of ethanyl, ethenyl, ethynyl, and triazinyl; R₁ is selected from the group of R₁ is selected from the group of alkyl, alkoxy, cycloalkyl, —CH₂-cycloalkyl, —O— cycloalkyl, halogen, haloalkyl, OH, and CN; and R₂ is a ring moiety selected from the group of imidazolyl, pyrazolyl, 1,2,3-triazolyl, thiazolyl, phenyl, and pyridinyl, each optionally substituted; for use as inhibitors against native BCR-ABL kinase protein and clinically important BCR-ABL mutations such as T315I, F317L, E255K and Y253F for the treatment of diseases that include chronic myeloid leukemia (CML), acute lymphoblastic leukemia (ALL), and acute myelogenous leukemia (AML).

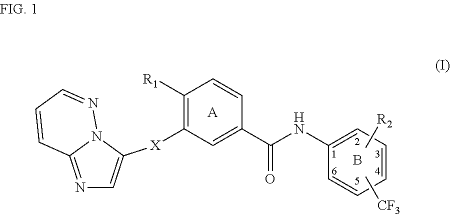

### CROSS-REFERENCE TO RELATED APPLICATIONS

This is the 371 National Phase of International Application No. PCT/US23/31451, filed Aug. 29, 2023; which claims priority under 35 U.S.C. § 119 to U.S. Provisional Patent Application No. 63/373,842, filed Aug. 29, 2022, which is hereby incorporated by reference in its entirety.

### STATEMENT REGARDING FEDERALLY SPONSORED RESEARCH OR DEVELOPMENT

This invention was made with government support under TR002369 awarded by the National Institutes of Health. The government has certain rights in the invention.

### FIELD OF THE INVENTION

The present invention concerns small molecule potent BCR-ABL inhibitors effective against native BCR-ABL kinase protein and clinically important BCR-ABL mutations such as T315I, F317L, E255K and Y253F.

### BACKGROUND OF THE INVENTION

Development of tyrosine kinase inhibitors (TKIs) targeting the BCR-ABL oncogene constitute an effective approach to treat chronic myeloid leukemia (CML) and/or acute lymphoblastic leukemia (ALL). Currently available inhibitors are limited by drug resistance and toxicity. Ponatinib a third-generation inhibitor has demonstrated excellent efficacy against both wild type and mutant BCR-ABL kinase, including the “gatekeeper” T315I mutation which is resistant to all other currently available TKIs. However, it is one of the most cardiotoxic of FDA-approved TKIs. Therefore, an ideal inhibitor should be effective against both native and clinically important BCR-ABL mutations and highly cardiac-safe and immune safe compared to ponatinib.

### SUMMARY OF THE INVENTION

A first embodiment provides a compound of Formula (I):

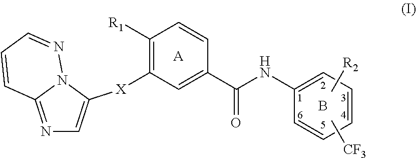

wherein:
X is selected from the group of:

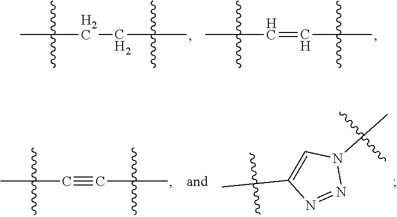

R₁ is selected from the group of C₁-C₆ alkyl, C₁-C₆ alkoxy, C₃-C₆ cycloalkyl, —CH₂—C₃-C₆ cycloalkyl, —O—C₃-C₆ cycloalkyl, halogen, C₁-C₃ haloalkyl, OH, and CN;
R₂ is selected from the group of H, —NH—S(═O)₂H, —NH—S(═O)₂—C₁-C₆ alkyl,

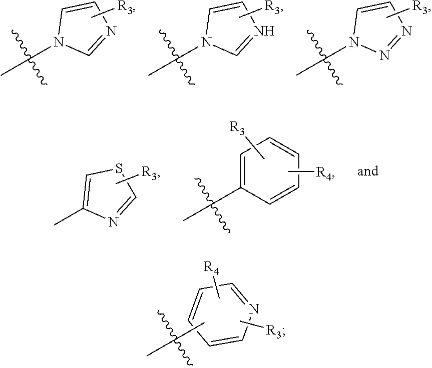

R₃ and R₄ are each independently selected from the group of H, C₁-C₆ alkyl, —O—C₁-C₆ alkyl, C₁-C₆ haloalkyl, —O—C₁-C₆ haloalkyl, halogen, —NH—S(═O)₂H, and —NH—S(═O)₂—C₁-C₆ alkyl;
with the proviso that, when X is

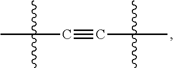 [SMILES: *C#C*]

R₁ is CH₃ or Cl, and the variable CF₃ group is bound to either position 3 or position 5 of phenyl ring B, then R₂ is not

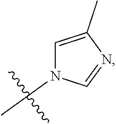 [SMILES: *n1cnc(C)c1]

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

### BRIEF DESCRIPTION OF THE MANY VIEWS OF THE DRAWINGS

FIG. 1 presents dose response curves of imatinib, ponatinib, and CET analogs CET-109, CET-110, CET-111, and CET-84 against Ba/F3 cells expressing BCR-ABL T315I.

FIG. 2 presents dose response curves of imatinib, ponatinib, and CET analogs CET-067, CET-084, CET-105, and CET-104 against Ba/F3 cells expressing BCR-ABL T315I.

FIG. 3 presents dose response curves of imatinib, ponatinib, and CET analogs CET-001, CET-104, CET-105, and CET-106 against Ba/F3 cells expressing BCR-ABL T315I.

FIG. 4 presents dose response curves of imatinib, ponatinib, and CET analogs CET-84, CET-99, and CET-100 against Ba/F3 cells expressing BCR-ABL T315I.

FIG. 5 presents dose response curves of imatinib, ponatinib, and CET analogs CET-100, CET-101, CET-102, and CET-103 against Ba/F3 cells expressing BCR-ABL T315I.

FIG. 6 presents dose response curves of imatinib, ponatinib, and CET analogs CET-67, CET-84, CET-85, CET-90, and CET-91 against Ba/F3 cells expressing BCR-ABL T315I.

FIG. 7 presents dose response curves of imatinib, ponatinib, and CET analogs CET-99, CET-100, CET-101, CET-102, and CET-103 against Ba/F3 cells expressing BCR-ABL T315I.

FIG. 8 presents dose response curves of imatinib, ponatinib, and CET analogs CET-84, CET-99, and CET-100 against Ba/F3 cells expressing BCR-ABL T315I.

FIG. 9 presents dose response curves of imatinib, ponatinib, and CET analogs CET-84, CET-85, CET-99, and CET-100 against Ba/F3 cells expressing BCR-ABL T315I.

FIG. 10 presents dose response curves of imatinib, ponatinib, and CET analogs CET-84, CET-99, and CET-100 against an HL60 AML-derived cell line.

FIG. 11 presents dose response curves of imatinib, ponatinib, and CET analogs CET-109, CET-110, CET-111, and CET-84 against Ba/F3 cells expressing BCR-ABL E255V.

FIG. 12 presents dose response curves of imatinib, ponatinib, and CET analogs CET-001, CET-004, CET-105, and CET-106 against a K562 CML-derived cell line.

FIG. 13 presents dose response curves of imatinib, ponatinib, and CET analogs CET-99, CET-100, CET-101, CET-102, and CET-103 against an AML2 AML-derived cell line.

FIG. 14 presents dose response curves of imatinib, ponatinib, and CET analogs CET-001, CET-004, CET-105, and CET-106 against an AML1 AML-derived cell line.

FIG. 15 presents dose response curves of imatinib, ponatinib, and CET analogs CET-84, CET-99, and CET-100 against an AML1 AML-derived cell line.

FIG. 16 presents dose response curves of imatinib, ponatinib, and CET analogs CET-001, CET-004, CET-105, and CET-106 against a K562 CML-derived cell line.

FIG. 17 presents dose response curves of imatinib, ponatinib, and CET analogs CET-84, CET-85, CET-90, and CET-100 against Ba/F3 cells expressing BCR-ABL E255V.

FIG. 18 presents dose response curves of imatinib, ponatinib, and CET analogs CET-84, CET-99, and CET-100 against Ba/F3 cells expressing BCR-ABL E255V.

FIG. 19 presents ¹³C NMR of N-(3-(1H-imidazol-1-yl)-5-(trifluoromethyl)phenyl)-3(imidazo[1,2-b]4yridazine-3-ylethynyl)-4-methylbenzamide (CET-10-084).

FIG. 20 presents 1H NMR of N-(3-(1H-imidazol-1-yl)-5-(trifluoromethyl)phenyl)-3-(imidazo[1,2-b]4yridazine-3-ylethynyl)-4-methylbenzamide (CET-10-084).

FIG. 21 presents ¹H NMR of 3-(imidazo[1,2-b]4yridazine-3-ylethynyl)-4-methyl-N-(3-(2-methyl-1H-imidazol-1-yl)-5-(trifluoromethyl)phenyl)benzamide (CET-10-067).

FIG. 22 presents ¹³C NMR of 3-(imidazo[1,2-b]4yridazine-3-ylethynyl)-4-methyl-N-(3-(2-methyl-1H-imidazol-1-yl)-5-(trifluoromethyl)phenyl)benzamide (CET-10-067).

FIG. 23 presents 1H NMR of N-(3-(4-fluoro-1H-imidazol-1-yl)-5-(trifluoromethyl)phenyl)-3-(imidazo[1,2-b]4yridazine-3-ylethynyl)-4-methylbenzamide (CET-10-100).

FIG. 24 presents ¹³C NMR of N-(3-(4-fluoro-1H-imidazol-1-yl)-5-(trifluoromethyl)phenyl)-3-(imidazo[1,2-b]4yridazine-3-ylethynyl)-4-methylbenzamide (CET-10-100).

FIG. 25 presents ¹H NMR of 3-(imidazo[1,2-b]4yridazine-3-ylethynyl)-4-methyl-N-(3-(trifluoromethyl)-5-(4-(trifluoromethyl)-1H-imidazol-1-yl)phenyl)benzamide (CET-10-103).

FIG. 26 presents ¹³C NMR of 3-(imidazo[1,2-b]4yridazine-3-ylethynyl)-4-methyl-N-(3-(trifluoromethyl)-5-(4-(trifluoromethyl)-1H-imidazol-1-yl)phenyl)benzamide (CET-10-103).

FIG. 27 presents ¹H NMR of 3-(imidazo[1,2-b]4yridazine-3-ylethynyl)-4-methyl-N-(5-(trifluoromethyl)-[1,1′-biphenyl]-3-yl)benzamide (CET-10-106).

FIG. 28 presents ¹³C NMR of 3-(imidazo[1,2-b]5yridazine-3-ylethynyl)-4-methyl-N-(5-(trifluoromethyl)-[1,1′-biphenyl]-3-yl)benzamide (CET-10-106).

FIG. 29 presents ¹H NMR of 3-(imidazo[1,2-b]5yridazine-3-ylethynyl)-4-methyl-N-(3-(pyridin-4-yl)-5-(trifluoromethyl)phenyl)benzamide (CET-10-104).

FIG. 30 presents ¹³C NMR of 3-(imidazo[1,2-b]5yridazine-3-ylethynyl)-4-methyl-N-(3-(pyridin-4-yl)-5-(trifluoromethyl)phenyl)benzamide (CET-10-104).

FIG. 31 presents ¹H NMR of N-(4-(1H-imidazol-1-yl)-3-(trifluoromethyl)phenyl)-3-(imidazo[1,2-b]5yridazine-3-ylethynyl)-4-methylbenzamide (CET-10-105).

FIG. 32 presents ¹³C NMR of N-(4-(1H-imidazol-1-yl)-3-(trifluoromethyl)phenyl)-3-(imidazo[1,2-b]5yridazine-3-ylethynyl)-4-methylbenzamide (CET-10-105).

FIG. 33 presents ¹H NMR of N-(3-(1-(difluoromethyl)-1H-pyrazol-4-yl)-5-(trifluoromethyl)phenyl)-3-(imidazo[1,2-b]5yridazine-3-ylethynyl)-4-methylbenzamide (CET-10-109).

FIG. 34 presents ¹³C NMR of N-(3-(1-(difluoromethyl)-1H-pyrazol-4-yl)-5-(trifluoromethyl)phenyl)-3-(imidazo[1,2-b]5yridazine-3-ylethynyl)-4-methylbenzamide (CET-10-109).

FIG. 35 presents ¹H NMR of 3-(imidazo[1,2-b]5yridazine-3-ylethynyl)-4-methyl-N-(3-(methylsulfonamido)-5-(trifluoromethyl)phenyl)benzamide (CET-10-110).

FIG. 36 presents ¹³C NMR of 3-(imidazo[1,2-b]5yridazine-3-ylethynyl)-4-methyl-N-(3-(methylsulfonamido)-5-(trifluoromethyl)phenyl)benzamide (CET-10-110).

FIG. 37 presents ¹H NMR of 3-(imidazo[1,2-b]5yridazine-3-ylethynyl)-4-methyl-N-(3-(2-methylthiazol-4-yl)-5-(trifluoromethyl)phenyl)benzamide (CET-10-111).

FIG. 38 presents ¹³C NMR of 3-(imidazo[1,2-b]5yridazine-3-ylethynyl)-4-methyl-N-(3-(2-methylthiazol-4-yl)-5-(trifluoromethyl)phenyl)benzamide (CET-10-111).

FIG. 39 presents ¹H NMR of 3-(2-(imidazo[1,2-b]5yridazine-3-yl)vinyl)-4-methyl-N-(5-(trifluoromethyl)-[1,1′-biphenyl]-3-yl)benzamide (CET-10-101).

FIG. 40 presents ¹³C NMR of 3-(2-(imidazo[1,2-b]5yridazine-3-yl)vinyl)-4-methyl-N-(5-(trifluoromethyl)-[1,1′-biphenyl]-3-yl)benzamide (CET-10-101).

FIG. 41 presents ¹H NMR of 3-(2-(imidazo[1,2-b]5yridazine-3-yl)vinyl)-4-methyl-N-(3-(pyridin-4-yl)-5-(trifluoromethyl)phenyl)benzamide (CET-10-102).

FIG. 42 presents ¹³C NMR of 3-(2-(imidazo[1,2-b]5yridazine-3-yl)vinyl)-4-methyl-N-(3-(pyridin-4-yl)-5-(trifluoromethyl)phenyl)benzamide (CET-10-102).

FIG. 43 presents 1H NMR of 3-(4-(imidazo[1,2-b]6yridazine-3-yl)-1H-1,2,3-triazol-1-yl)-4-methyl-N-(3-(4-methyl-1H-imidazol-1-yl)-5-(trifluoromethyl)phenyl)benzamide (CET-10-108).

FIG. 44 presents ¹³C NMR of 3-(4-(imidazo[1,2-b]6yridazine-3-yl)-1H-1,2,3-triazol-1-yl)-4-methyl-N-(3-(4-methyl-1H-imidazol-1-yl)-5-(trifluoromethyl)phenyl)benzamide (CET-10-108).

FIG. 45 presents 1H NMR of N-(3-(1H-imidazol-1-yl)-5-(trifluoromethyl)phenyl)-3-(4-(imidazo[1,2-b]6yridazine-3-yl)-1H-1,2,3-triazol-1-yl)-4-methylbenzamide (CET-10-107).

FIG. 46 presents ¹³C NMR of N-(3-(1H-imidazol-1-yl)-5-(trifluoromethyl)phenyl)-3-(4-(imidazo[1,2-b]6yridazine-3-yl)-1H-1,2,3-triazol-1-yl)-4-methylbenzamide (CET-10-107).

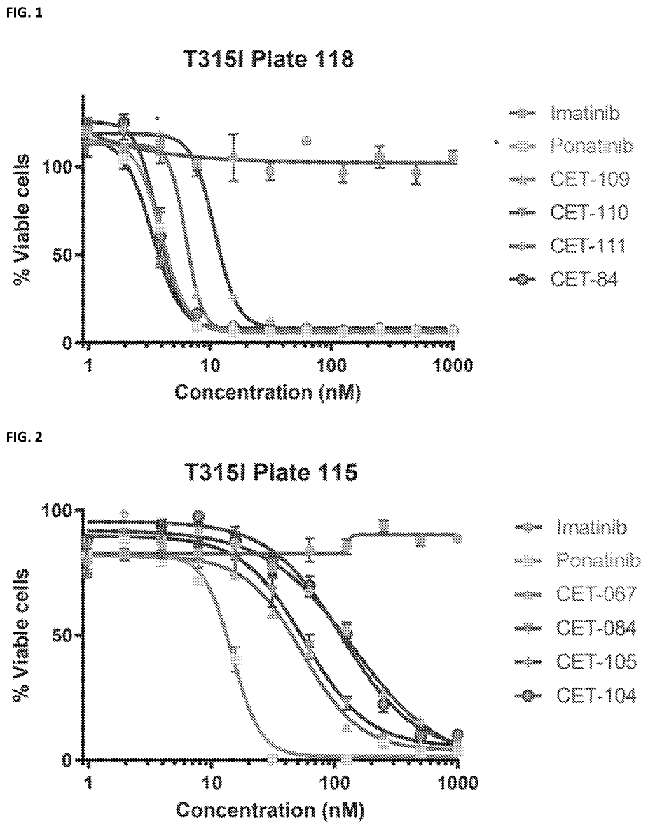

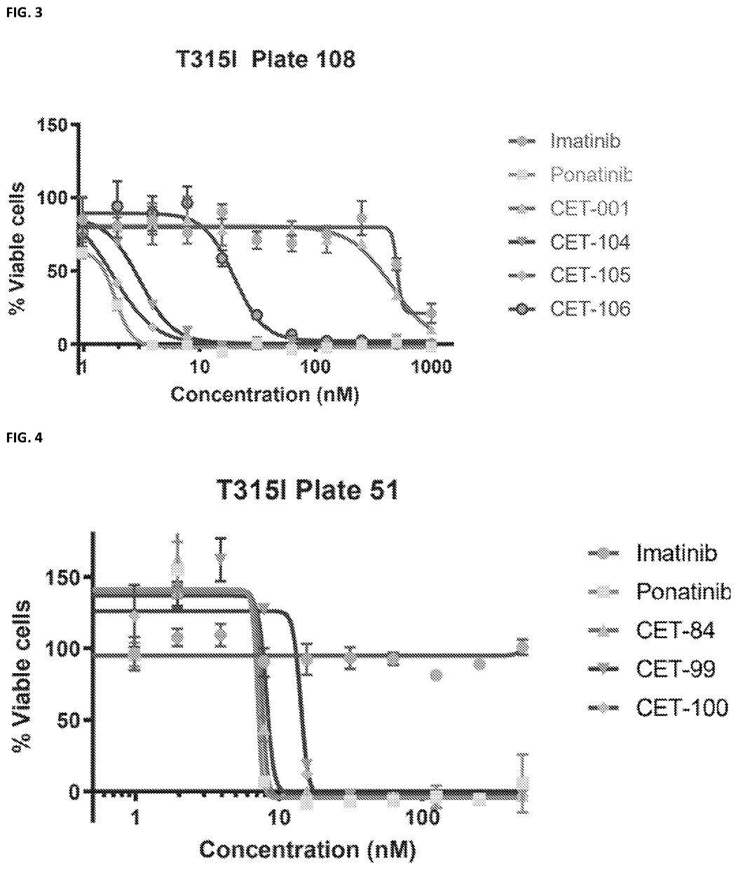

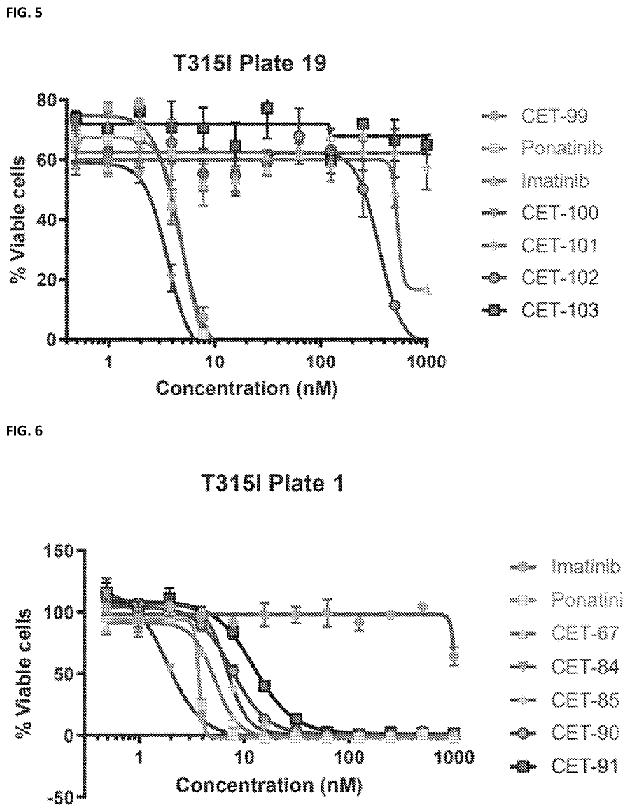

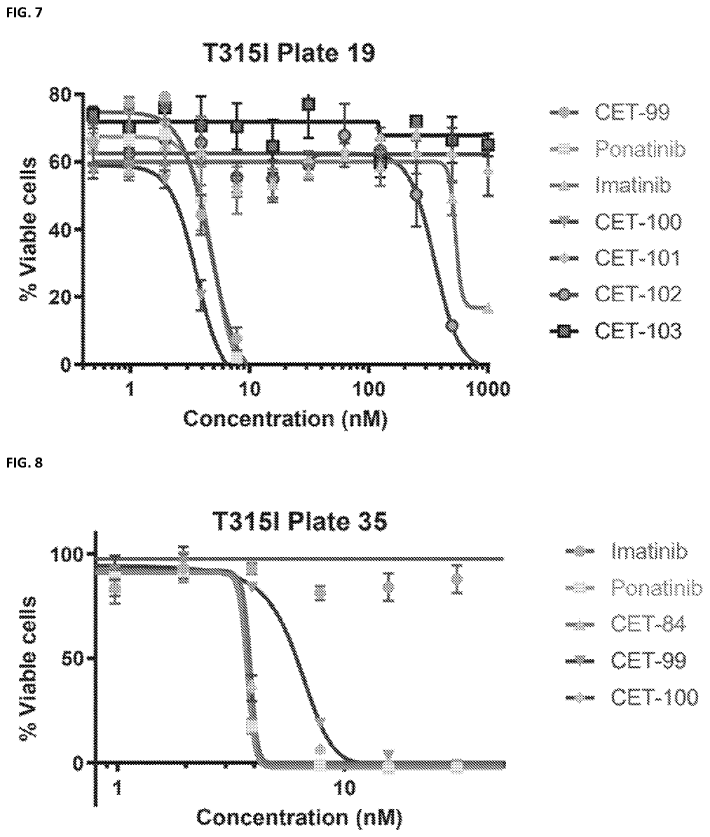

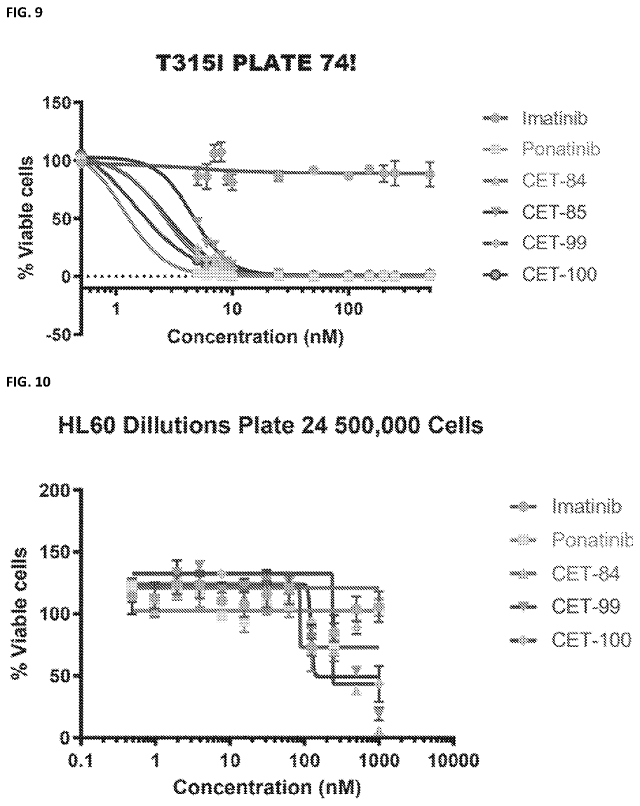

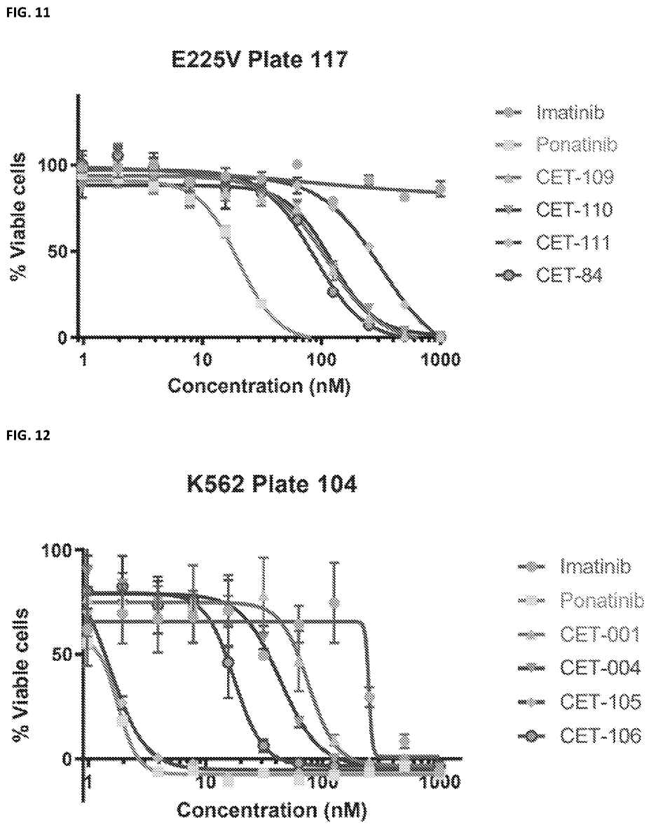

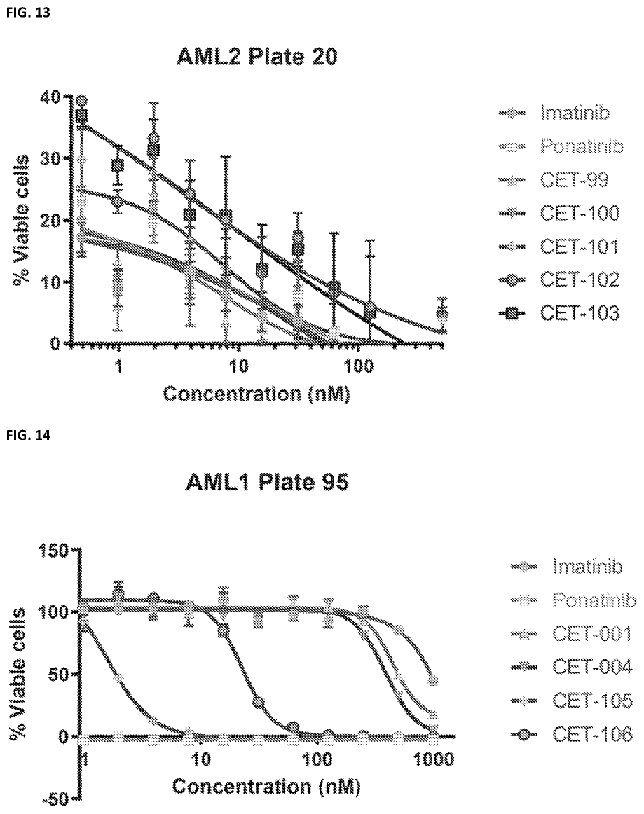

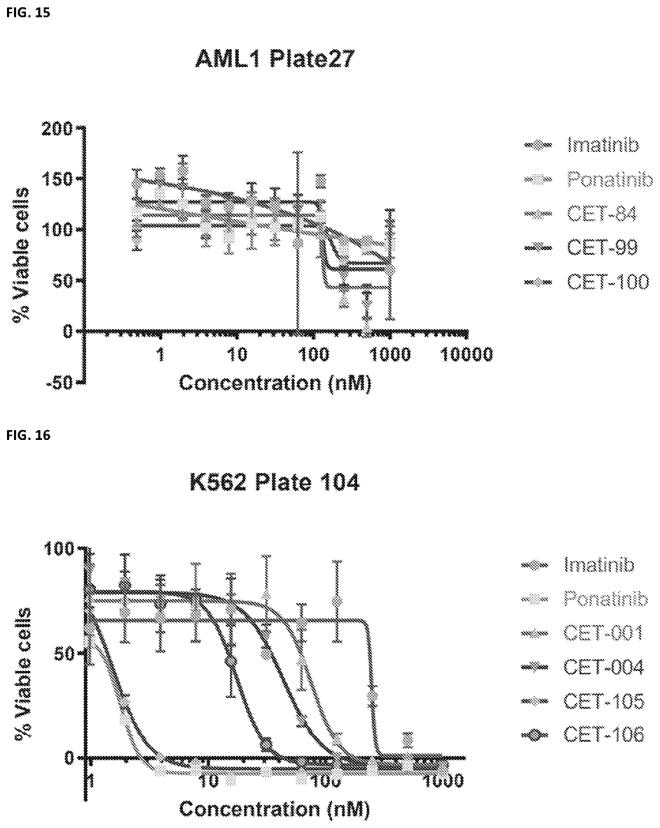

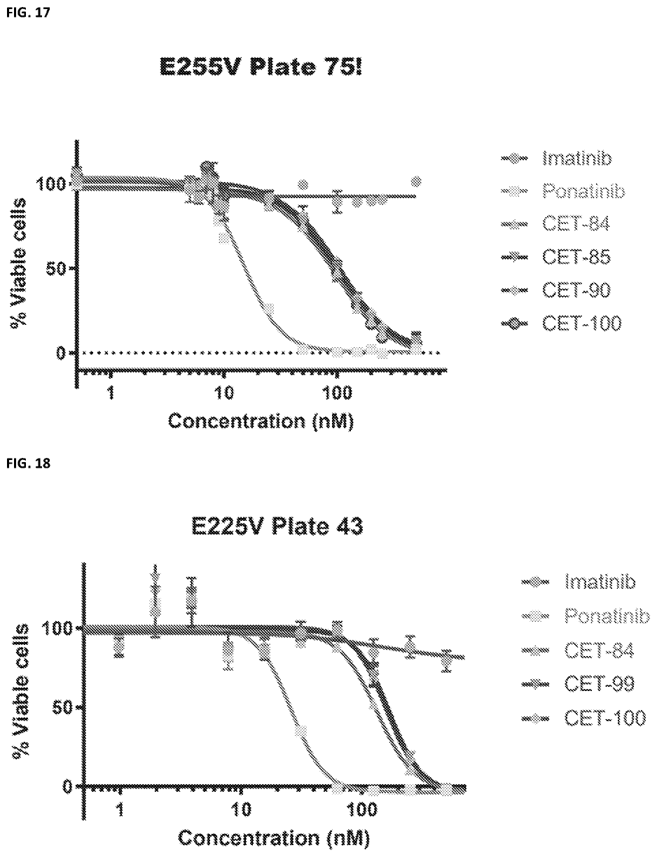

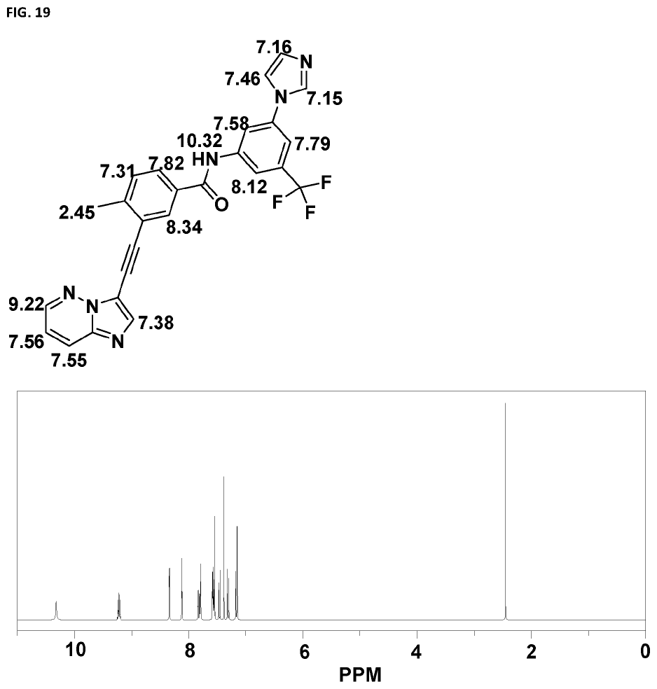

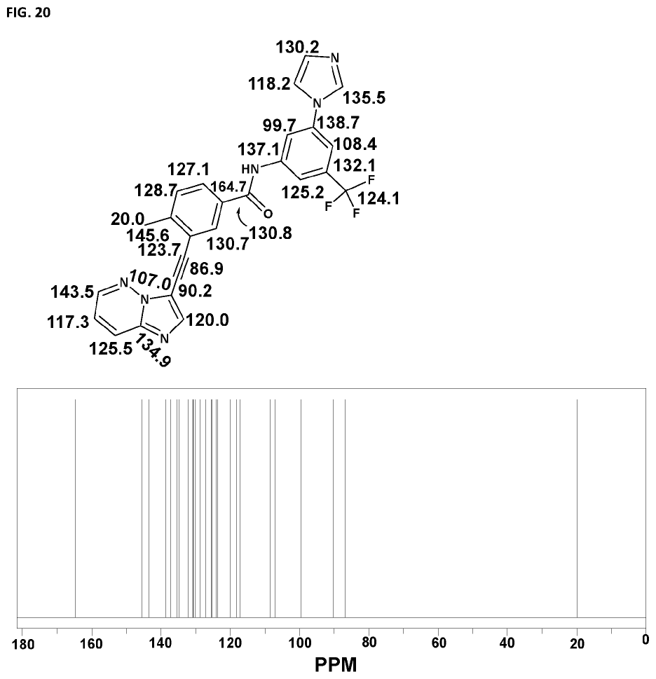

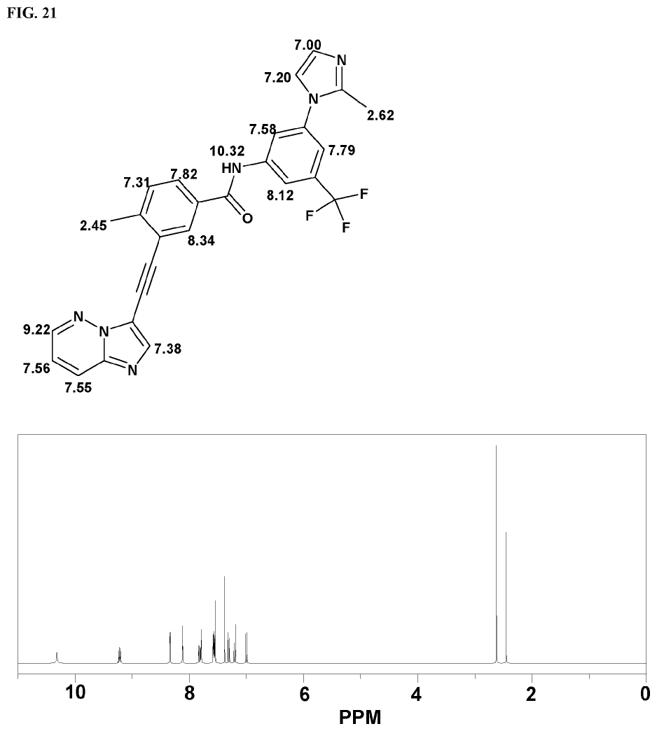

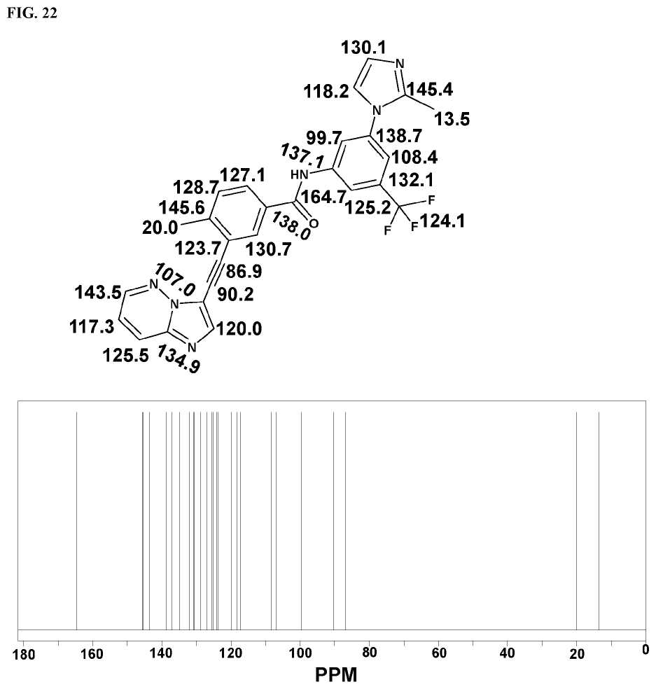

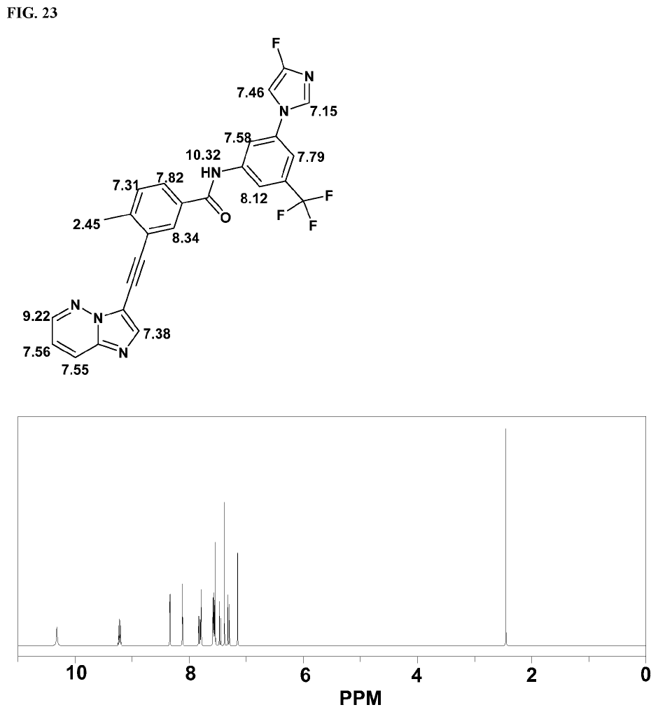

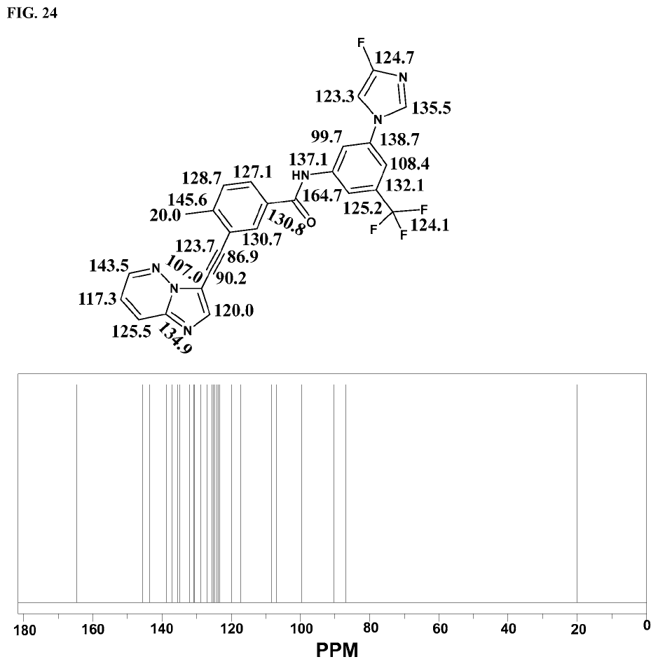

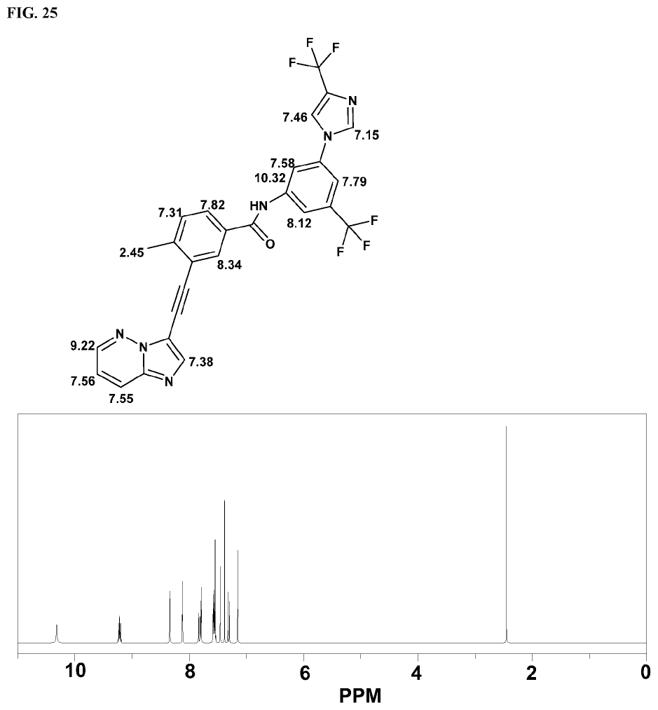

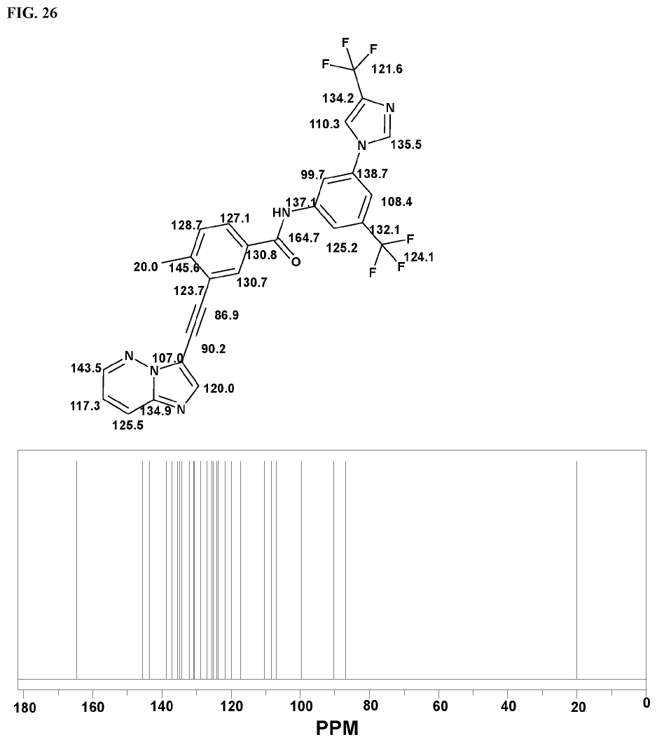

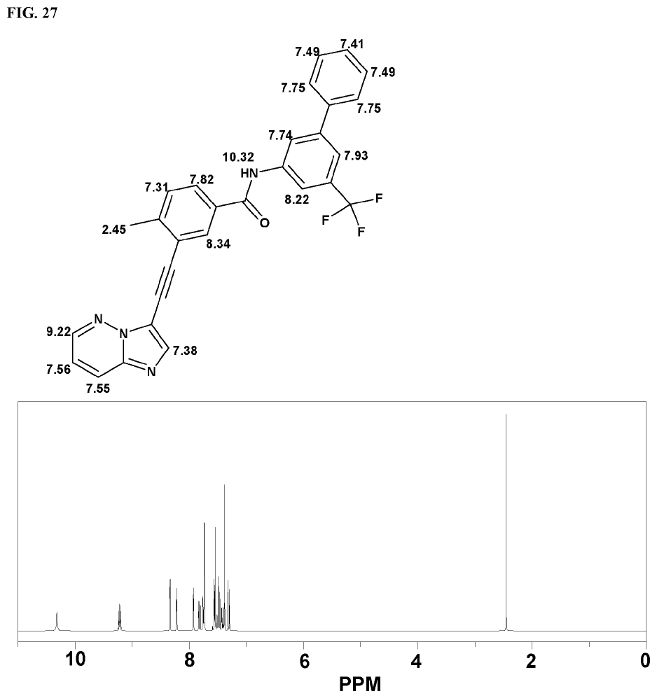

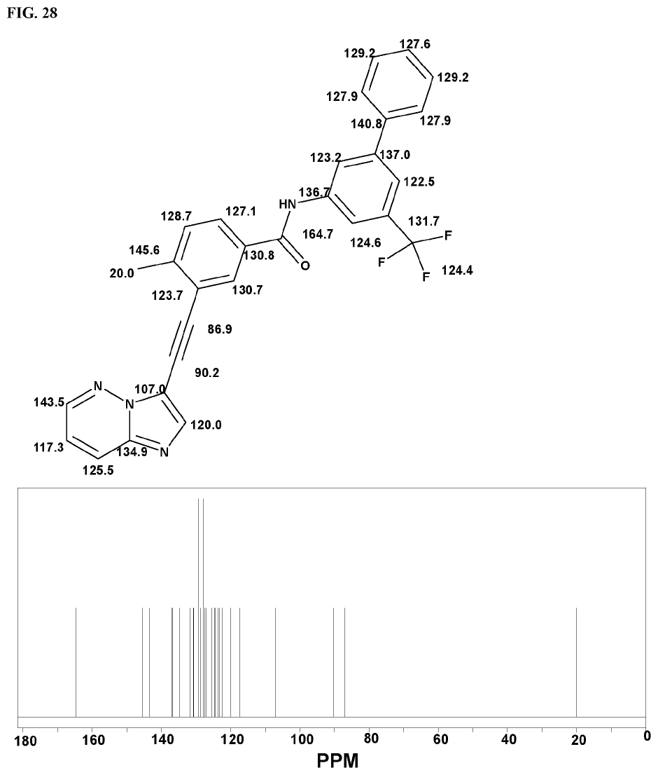

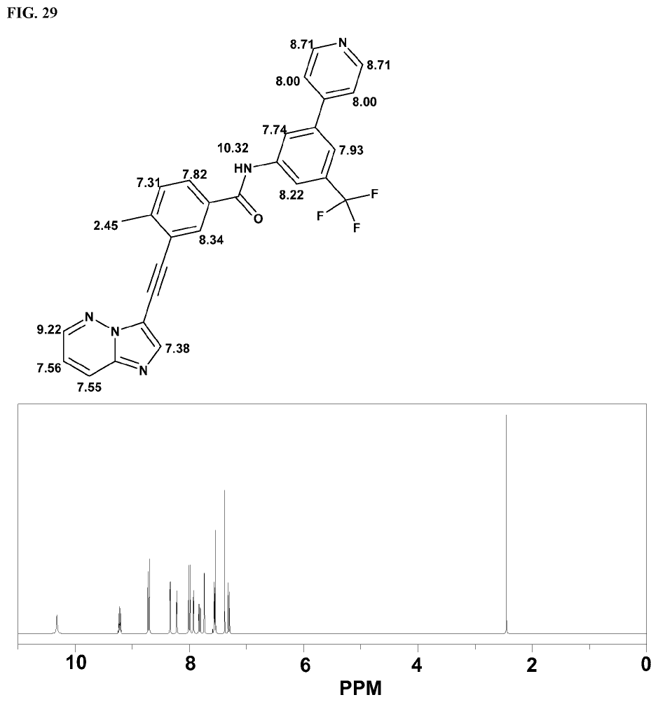

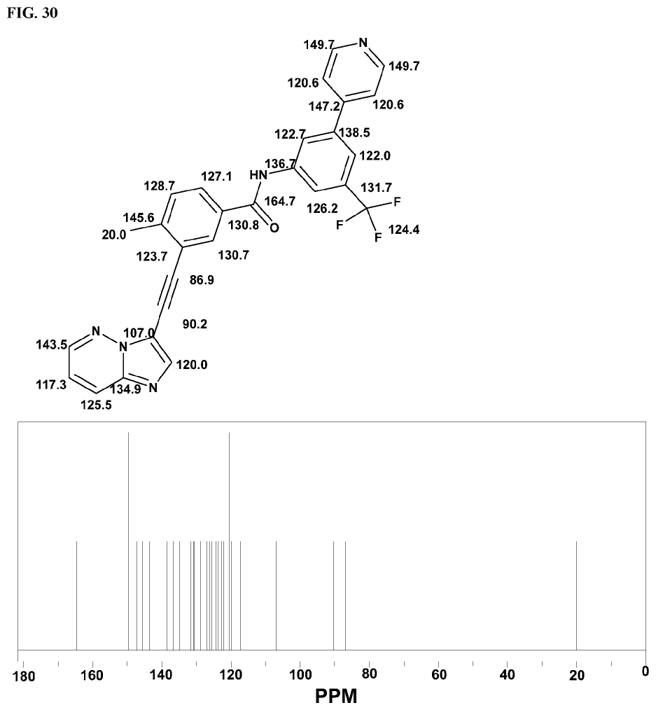

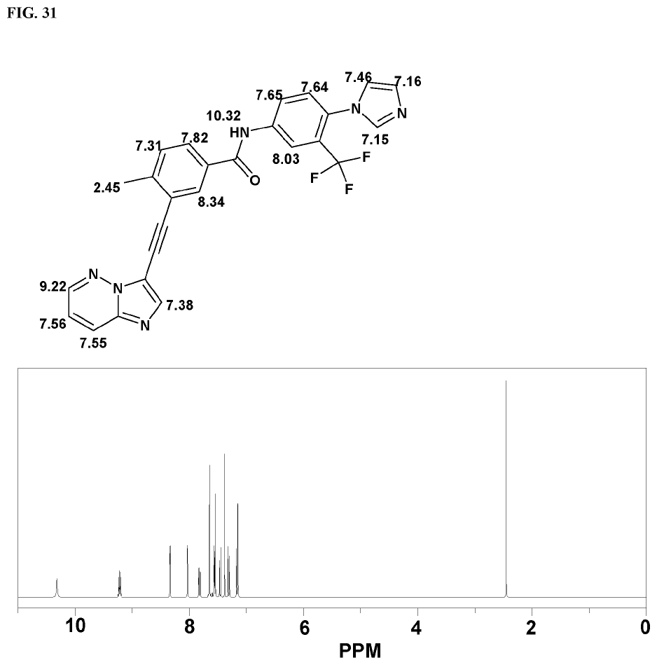

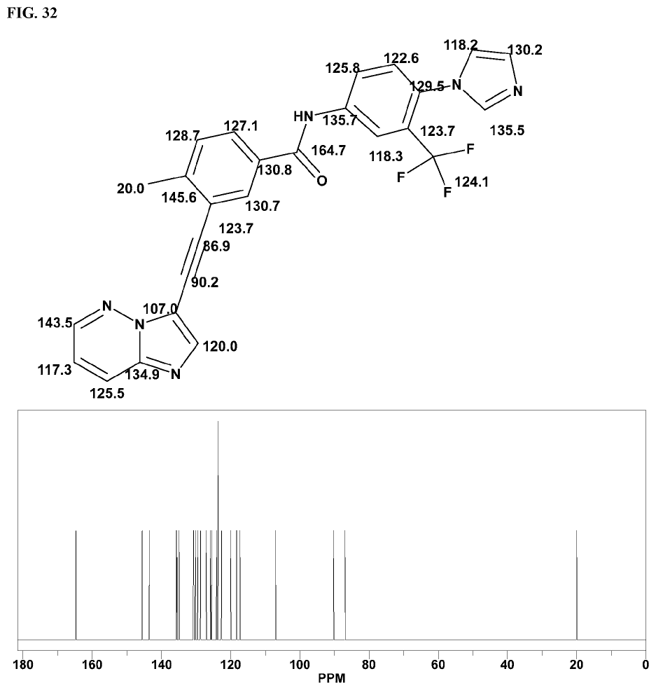

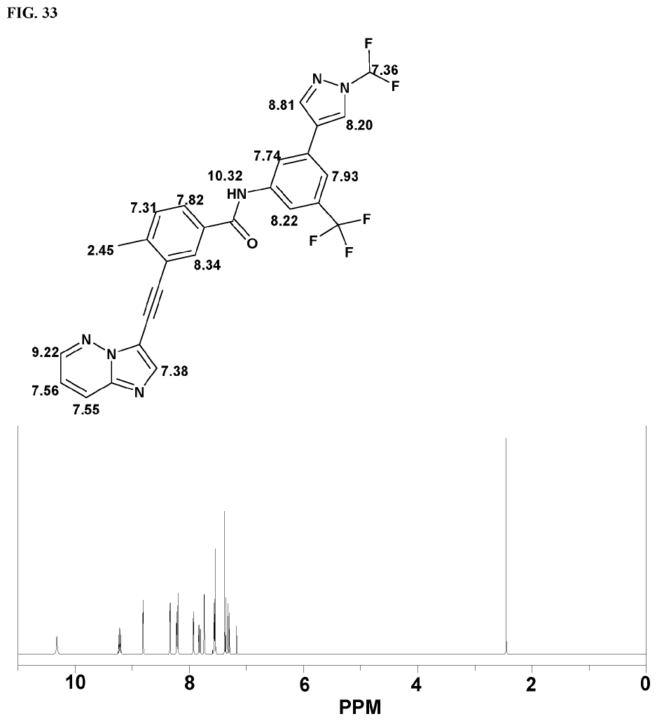

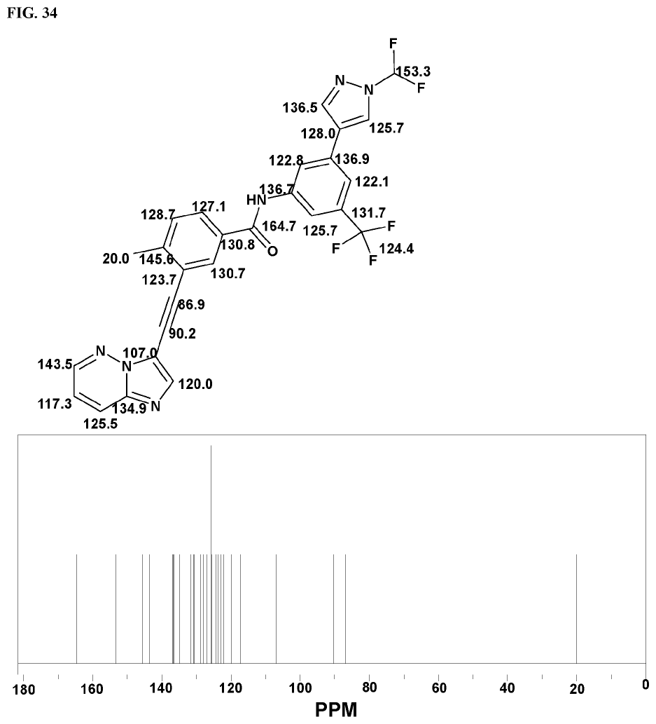

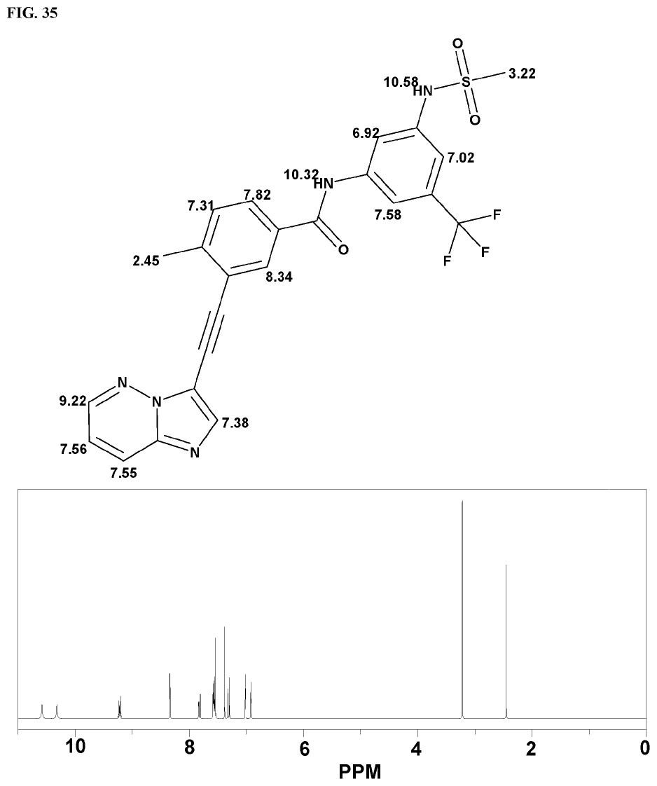

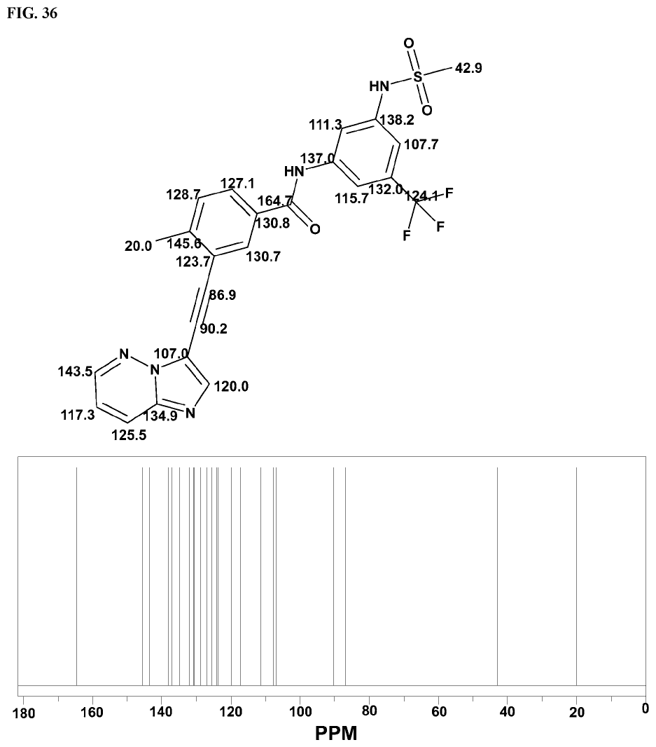

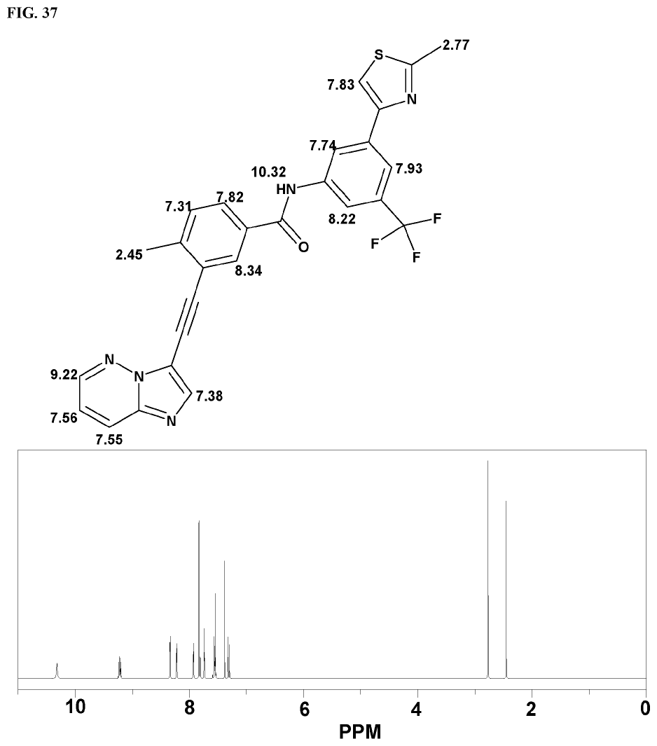

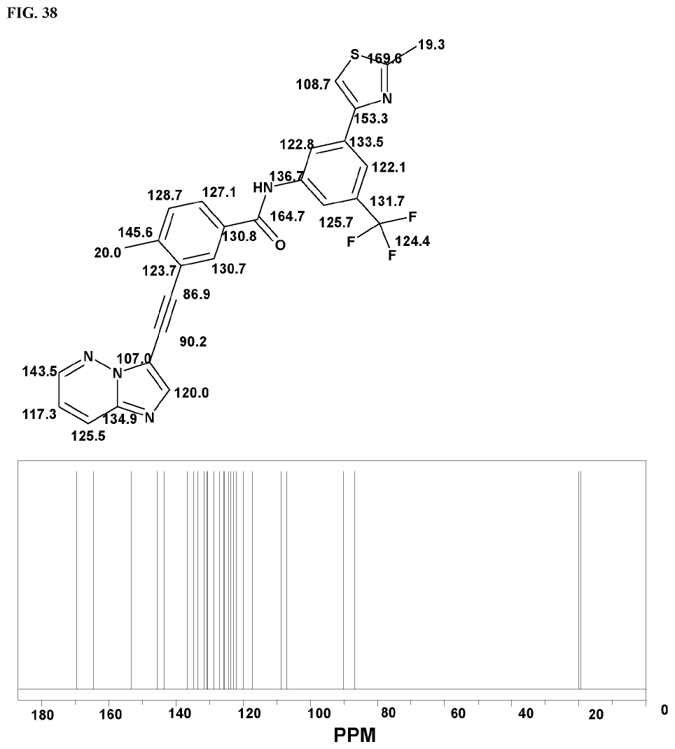

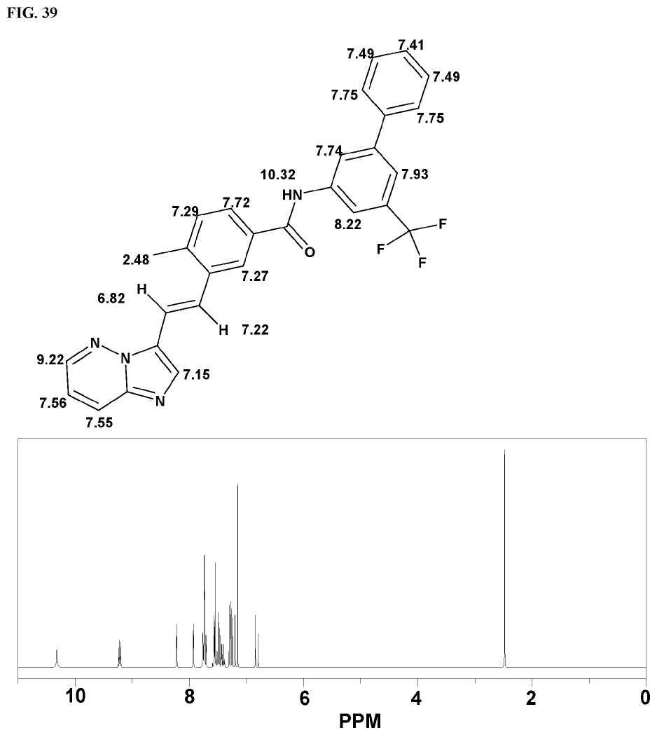

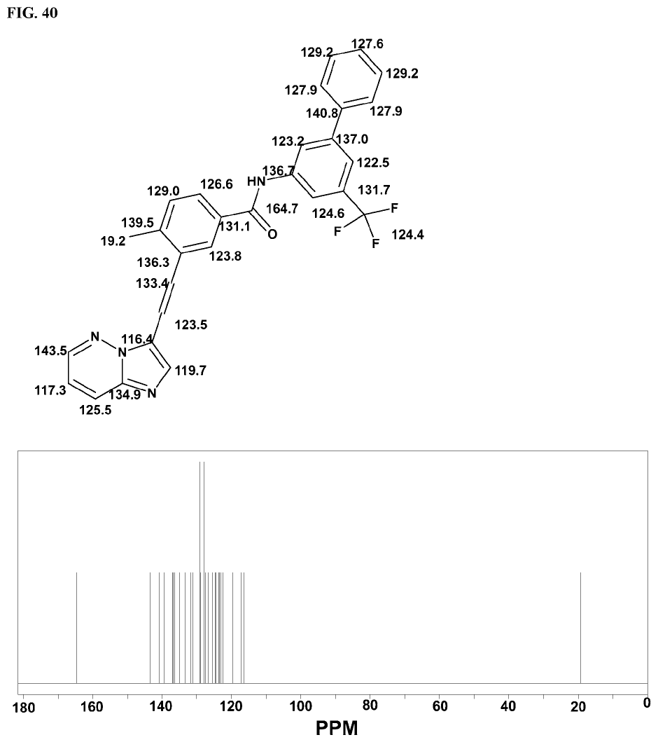

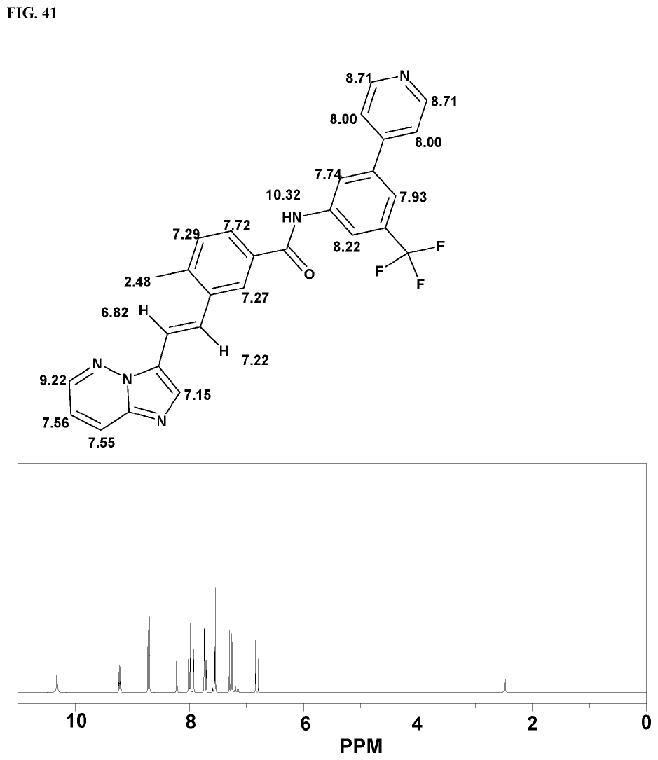

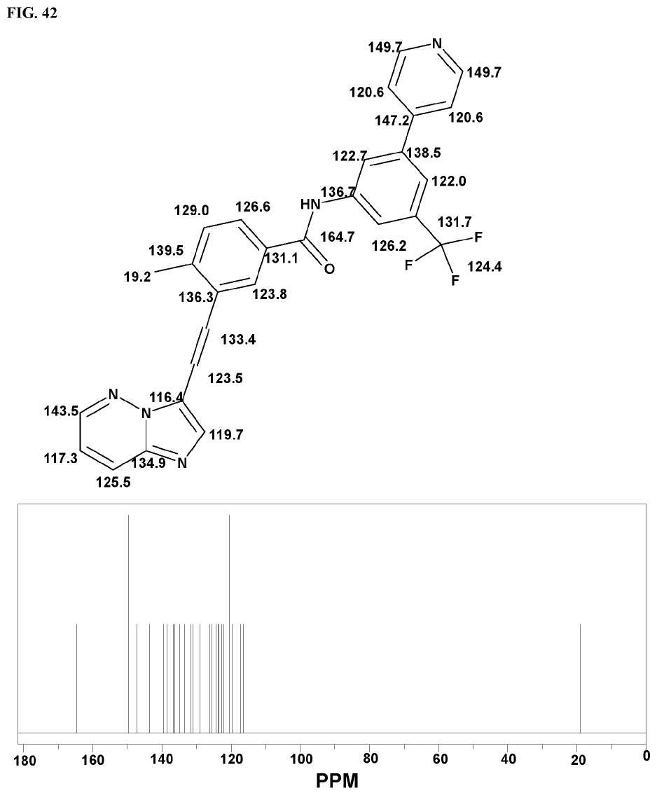

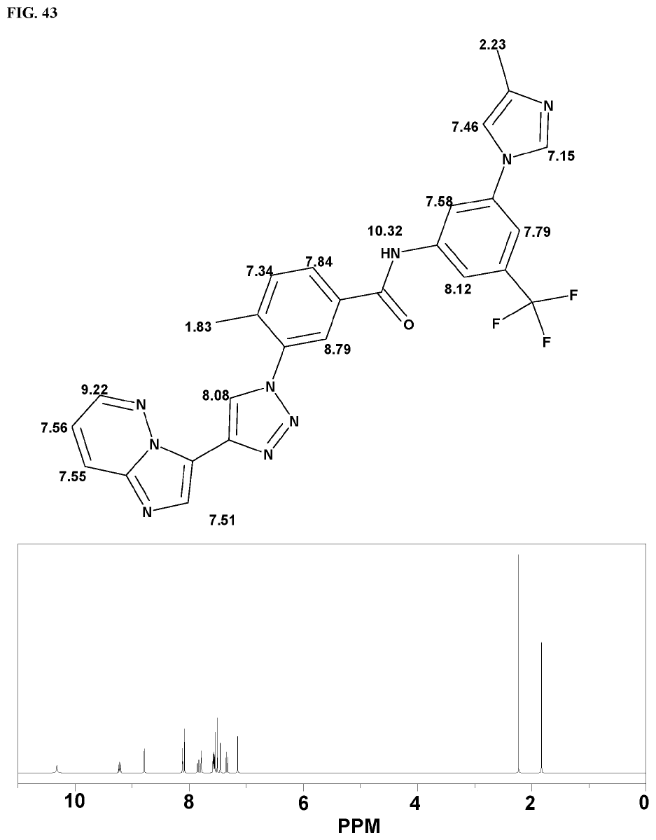

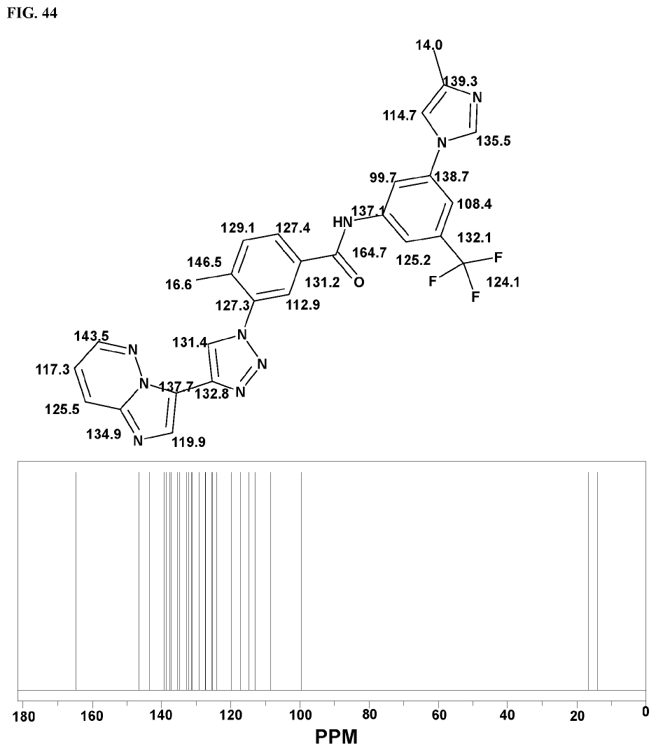

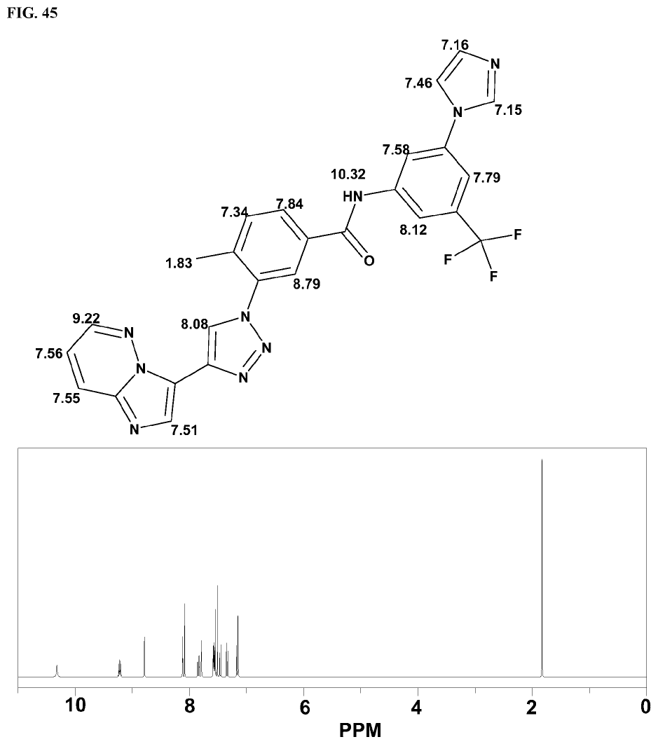

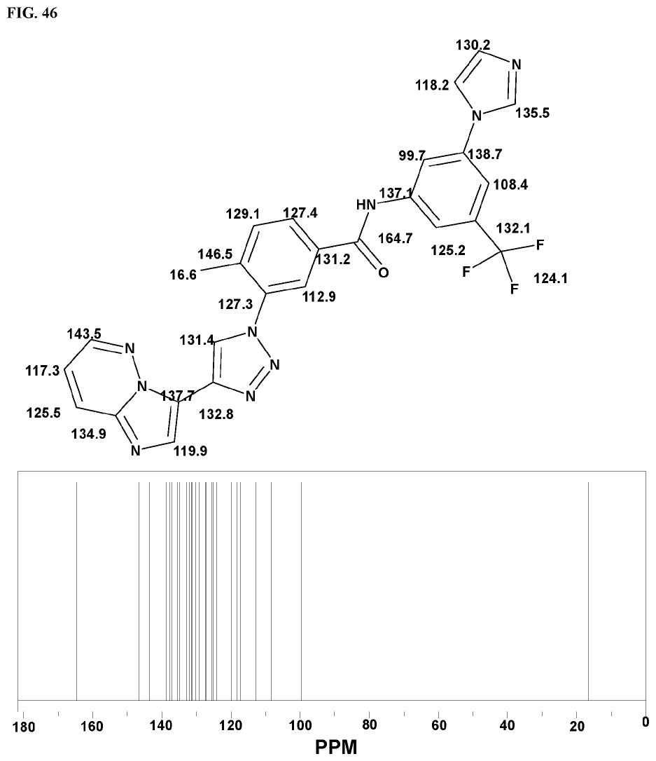

### DETAILED DESCRIPTION OF THE INVENTION

It is understood that the provisos in the first embodiment above exclude the compounds:
a) 3-(imidazo[1,2-b]pyridazin-3-ylethynyl)-4-methyl-N-(4-(4-methyl-1H-imidazol-1-yl)-3-(trifluoromethyl)phenyl)benzamide (CAS 1542265-39-3);
b) 3-(imidazo[1,2-b]pyridazin-3-ylethynyl)-4-methyl-N-(3-(4-methyl-1H-imidazol-1-yl)-5-(trifluoromethyl)phenyl)benzamide (CAS 1289555-64-1);
c) 4-chloro-3-(imidazo[1,2-b]pyridazin-3-ylethynyl)-N-(4-(4-methyl-1H-imidazol-1-yl)-3-(trifluoromethyl)phenyl)benzamide (CAS 1542265-40-6); and
d) 4-chloro-3-(imidazo[1,2-b]pyridazin-3-ylethynyl)-N-(3-(4-methyl-1H-imidazol-1-yl)-5-(trifluoromethyl)phenyl)benzamide (CAS 1542265-41-7).

Additional embodiments provide three separate compounds, respectively, of Formula (Ia), Formula (Ib), and Formula (Ic):

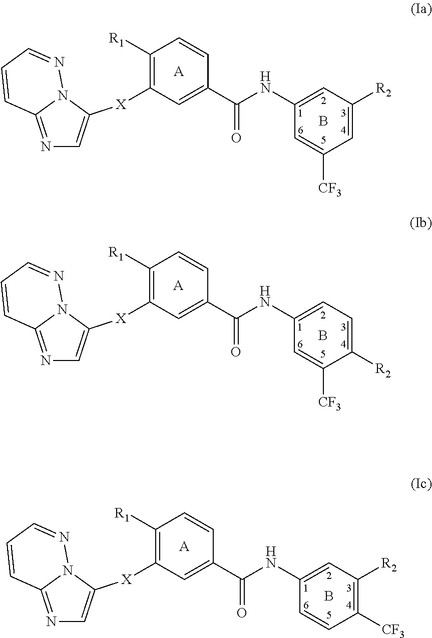

wherein, in each instance, all variables, including X, R₁, R₂, R₃, and R₄ are as defined, including provisos, for the embodiment of Formula (I), above, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Three additional separate embodiments provide, respectively, a compound of Formula (IIa), a compound of Formula (IIb), and a compound of Formula (IIc):

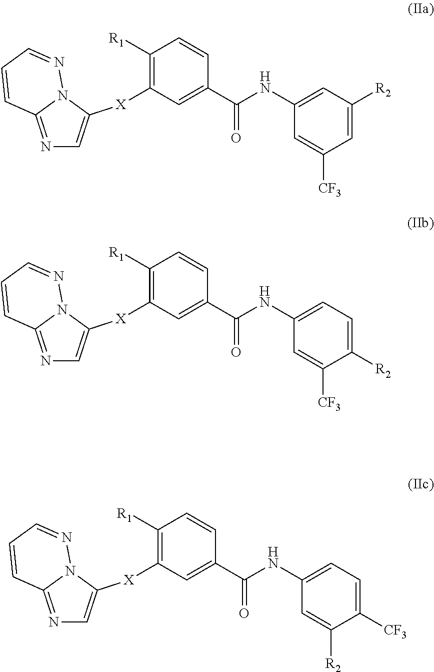

wherein, in each separate embodiment:
X is selected from the group of:

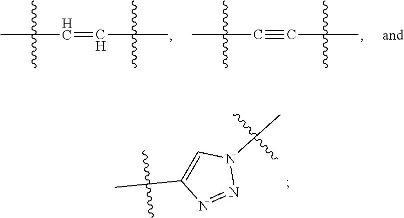

R₁ is selected from the group of C₁-C₄ alkyl, C₁-C₄ alkoxy, C₃-C₆ cycloalkyl, —CH₂—C₃-Cₑ cycloalkyl, —O-C₃-C₆ cycloalkyl, halogen, C₁-C₃ haloalkyl, OH, and ON;
   R₂ is selected from the group of H, —NH—S(═O)₂H, —NH—S(═O)₂—C₁-Cₑ alkyl,

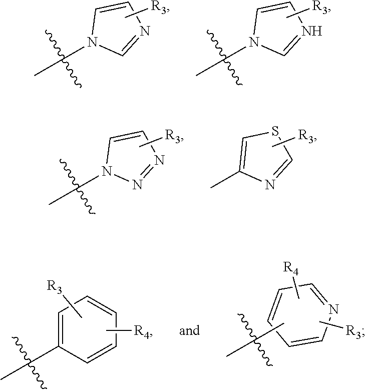

R₃ and R₄ are each independently selected from the group of H, C₁-C₆ alkyl, —O—C₁-C₆ alkyl, C₁-C₆ haloalkyl, halogen, —NH—S(═O)₂H, and —NH—S(═O)₂—C₁-C₆ alkyl;
with the proviso that, when X is

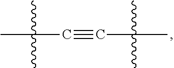 [SMILES: *C#C*]

R₁ is CH₃ or Cl, and the variable CF₃ group is bound to either position 3 or position 5 of phenyl ring B, then R₂ is not

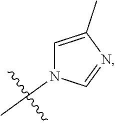 [SMILES: *n1cnc(C)c1]

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Three more separate embodiments provide, respectively, a compound of Formula (IIIa), a compound of Formula (IIIb), a compound of Formula (IIIc), a compound of Formula (IIIc), a compound of Formula (IIIe), a compound of Formula (IIIf), a compound of Formula (IIIg), a compound of Formula (IIIh), and a compound of Formula (IIIi):

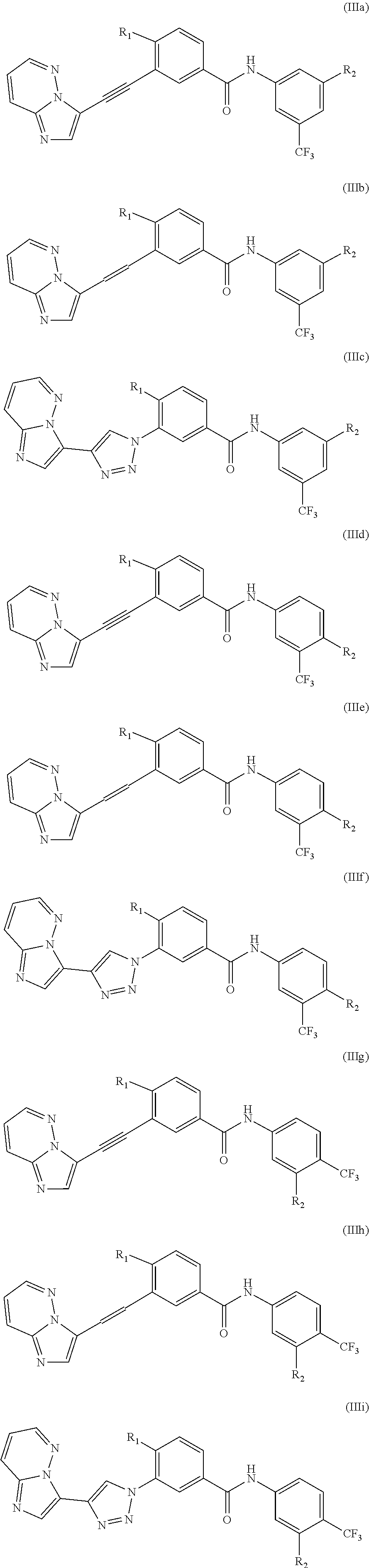

wherein, in each instance, R₁ and R₂ are as defined above for the embodiment(s) concerning a compound of Formula (IIa), a compound of Formula (IIb), and a compound of Formula (IIc); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Separate embodiments provide, respectively, a compound of each of Formulas (IVa) to (IVi):

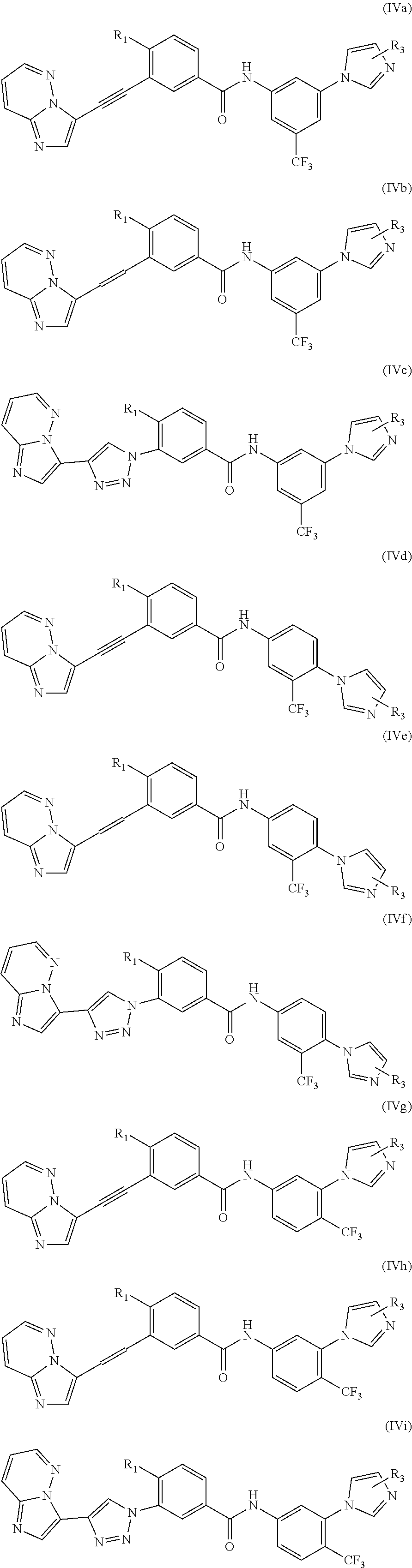

wherein, in each instance, R¹ and R₃ are as defined above for the embodiment(s) concerning a compound of Formulas (IIa)-(IIc); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Separate embodiments provide, respectively, a compound of each of Formulas (Va) to (Vi):

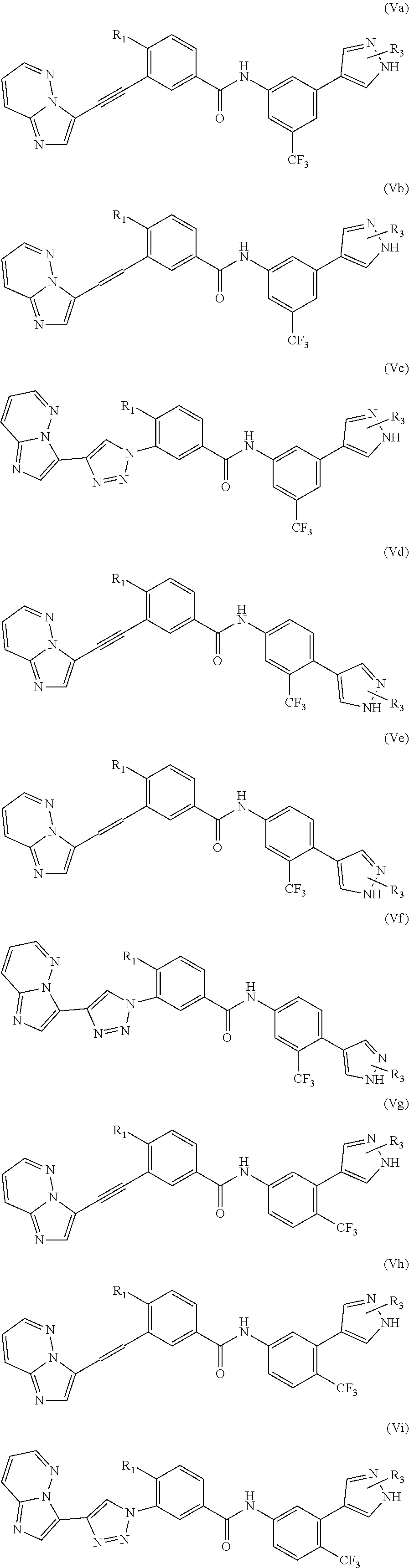

wherein, in each instance, R₁ and R₃ are as defined above for the embodiment(s) concerning a compound of Formulas (IIa) to (IIc); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Further embodiments provide, respectively, a compound of each of Formulas (VIa) to Formula (VIi):

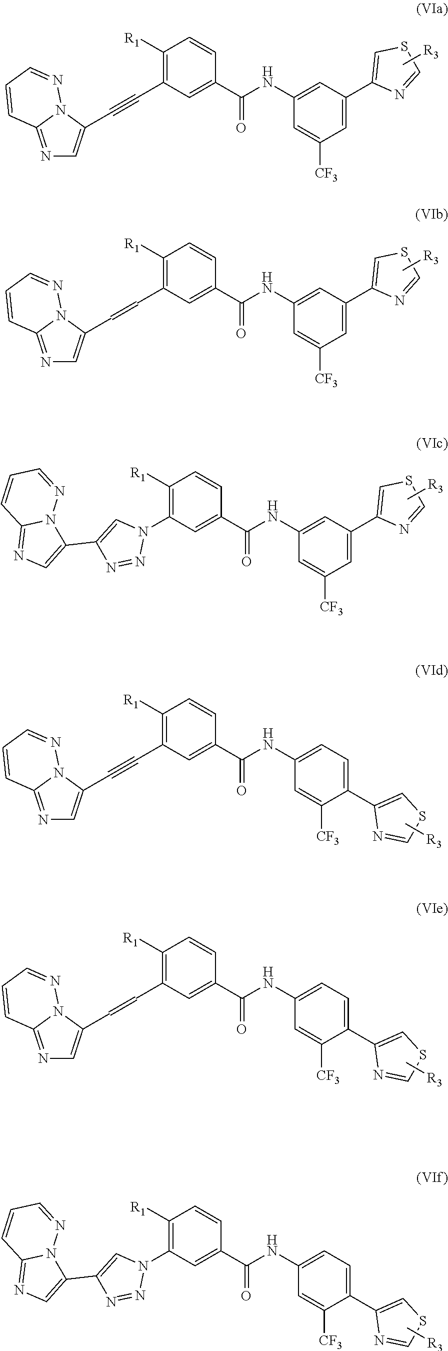

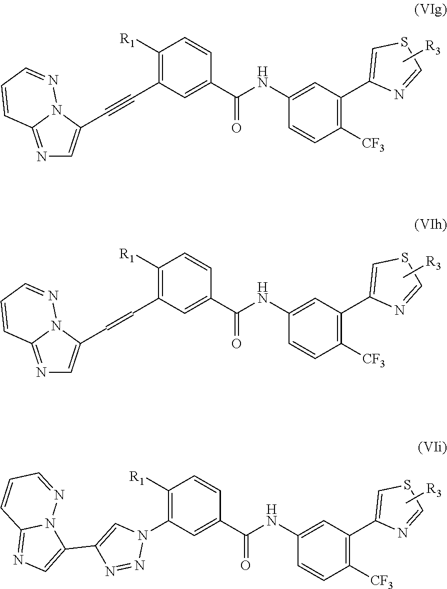

wherein, in each instance, R₁ and R₃ are as defined above for the embodiment(s) concerning a compound of Formulas (IIa) to (IIb); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

More embodiments provide, respectively, a compound of Formulas (VIIa) to (VIIi):

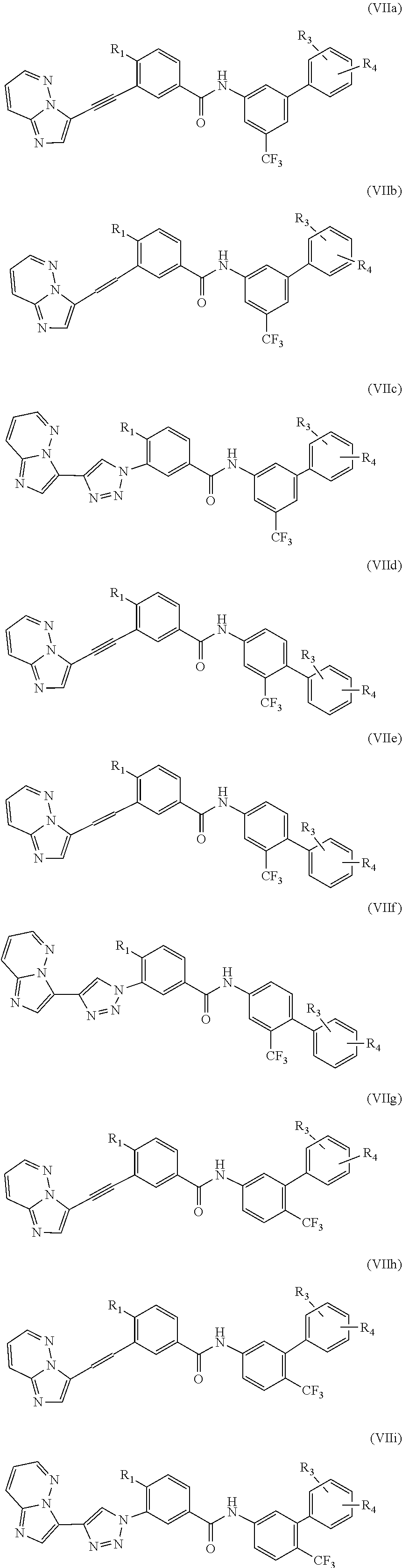

wherein, in each instance, R₁, R₃ and R₄ are as defined above for the embodiment(s) concerning a compound of Formulas (IIa) to (IIc); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Additional separate embodiments provide, respectively, a compound of Formulas (VIIIa) to (VIIIi):

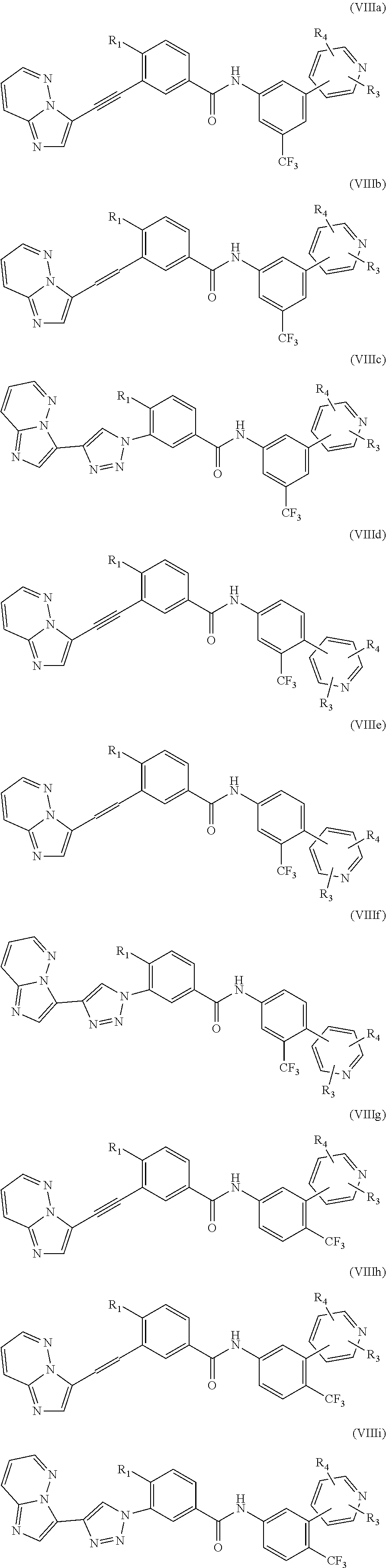

wherein, in each instance, R₁, R₃ and R₄ are as defined above for the embodiment(s) concerning a compound of Formulas (IIa) to (IIc); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

It is understood that different additional embodiments provide the corresponding compounds to those of Formulas (VIIIh) to (VIIIi) in which the pyridine ring substituted by R₃ and R₄ is, respectively, a 2-pyridinyl group, a 3-pyrdinyl group, and a 4-pyridinyl group, as indicated below:

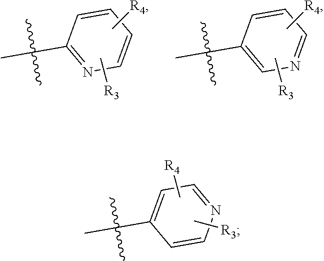

or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

### Definitions

The term “alkyl” refers to a straight or branched hydrocarbon. For example, an alkyl group can have 1 to 6 carbon atoms (i.e., C₁-C₆ alkyl or C₁₋₆ alkyl), 1 to 4 carbon atoms (i.e., C₁-C₄ alkyl or C₁₋₄ alkyl), or 1 to 3 carbon atoms (i.e., C₁-C₃ alkyl or C₁₋₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

The term “halogen” or “halo” refers and element or substituent selected from the group of F, CI, Br, and I.

The term “haloalkyl” refers to an alkyl group, as defined above, in which one or more hydrogen atoms of the alkyl group is replaced with a halogen atom. The alkyl portion of a haloalkyl group can have, for instance, 1 to 4 carbon atoms (i.e., C₁-C₄ haloalkyl), 1 to 3 carbon atoms (i.e., C₁-C₃ haloalkyl), or 1 to 2 carbon atoms (i.e., C₁-C₂ haloalkyl). Non-limiting examples of suitable haloalkyl groups, which may also be referred to as halofluoro groups, include, but are not limited to, trifluoromethyl (—CF₃), difluoromethyl (—CHF₂), fluoromethyl (—CFH₂), 2-fluoroethyl (—CH₂CH₂F), 2-fluoropropyl (—CH₂CHF₂), 2,2,2-trifluoroetheyl (—CH₂CF₃), 1,1-difluoroethyl (—CF₂CH₃), 2-fluoropropyl (—CH₂CHFCH₃), 1,1-difluoropropyl (—CF₂CH₂CH₃), 2,2-difluoropropyl (—CH₂CF₂CH₃), 3,3-difluoropropyl (—CH₂CH₂CHF₂), 3,3,3-trifluoropropyl (—CH₂CH₂CHF₃), 1,1-difluorobutyl (—CF₂CH₂CH₂CH₃), perfluoroethyl (—CF₂CF₃), perfluoropropyl (—CF₂CF₂CF₃), perfluoroproan-2-yl (—CF(CF₃)₂), 1,1,2,2,3,3-hexafluorobutyl (—CF₂—CF₂CF₂CH₃), perfluorobutyl (—CF₂CF₂CF₂CF₃), 1,1,1,3,3,3-hexafluoropropan-2-yl (—CH₂(CF₃)₂) groups, and the like. Additional groups wherein the halogen substitution is with bromine, iodine, or chlorine atoms are also understood for use herein.

The wavy line ( [symbol] ) in chemical structures indicates a bond through which the structure shown is bound to another chemical moiety or group.

The terms “treatment” or “treating” herein refer an approach for obtaining beneficial or desired results including clinical results. Beneficial or desired clinical results may include one or more of the following: (i) inhibiting the disease or condition (e.g., decreasing one or more symptoms resulting from the disease or condition (including ALL, CLL, CML, and the other diseases or disorders described herein), and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicates a decrease, such as a significant decrease, in the baseline activity of a biological activity or process. “Inhibition of BCR-ABL activity” refers to a decrease in BCR-ABL activity as a direct or indirect response to the presence of a compound of Formula I, or a pharmaceutically acceptable salt or co-crystal thereof, relative to the activity of BCR-ABL in the absence of such compound or a pharmaceutically acceptable salt or co-crystal thereof. The decrease in activity may be due to the direct interaction of the compound with BCR-ABL, or due to the interaction of the compound(s) described herein with one or more other factors that in turn affect BCR-ABL activity. For example, the presence of the compound(s) may decrease BCR-ABL activity by directly binding to the BCR-ABL, by causing (directly or indirectly) another factor to decrease BCR-ABL activity, or by (directly or indirectly) decreasing the amount of BCR-ABL present in the cell or organism. In some embodiments, the inhibition of BCR-ABL activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibition activity desired.

“Delaying” refers to the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given timeframe and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

The terms “subject” or “patient” refer to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example, treatment with a compound of Formula I, or a pharmaceutically acceptable salt or co-crystal thereof, as described herein. This includes a subject who may be determined to be at risk of or susceptible to such diseases or conditions, such that treatment would prevent the disease or condition from developing.

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

The terms “embodiment” and “aspect” used herein each refer to an example, an instance, or an illustration of the present disclosure and may be used interchangeably. In some instances, as described, one may further define, limit, or serve as a subset, subgeneric description, or specific example of the other. In other instances, one embodiment or aspect may provide a comparison to another or a distinction from the other.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

Variables such as X and R, including all subvariables thereof (such as R₁, R₂, etc.) used throughout the disclosure are the same variables as previously defined unless stated to the contrary.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

The term “independently selected” refers to a situation when more than one variable or item may be selected from a list of options regardless of which of the options applies to another variable or item. For instance, when variables R₃ and R₄ may be independently selected in each instance from the group of H, C₁-C₆ alkyl, —O-C₁-C₆ alkyl, C₁-C₆ haloalkyl, halogen, —NH—S(═O)₂H, and —NH—S(═O)₂—C₁-C₆ alkyl, R₃ and R₄ may each comprise the same option from the list (i.e. R₁ is —CH₃ and R₂ is —CH₃) or different options from the list (i.e. R₃ is —CH₃ and R₄ is H).

The term “therapeutically effective amount” or “pharmaceutically effective amount” refers to an amount or dose that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula I, or a pharmaceutically acceptable salt or co-crystal thereof, is an amount sufficient to modulate BCR-ABL expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of BCR-ABL activity.

In some embodiments, each dosage unit contains from 0.1 mg to 1 g, 0.1 mg to 500 mg, or 0.1 mg to 100 mg of a compound of Formula I, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof. In some embodiments, a therapeutically effective amount or a pharmaceutically effective amount of a compound of Formula (I), or a pharmaceutically acceptable salt thereof, comprises from about 0.1 mg to about 500 mg per dose, given once or twice daily. In some embodiments, the individual dose is selected from 1 mg, 5 mg, 10 mg, 20 mg, 30 mg, 40 mg, 50 mg, 60 mg, 75 mg, 100 mg, 150 mg, 200 mg, 250 mg, 300 mg, 350 mg, 400 mg, and 500 mg per dose. In some embodiments, an initial dose of from about 10 mg to about 100 mg may be used, with a reduction to lower dose upon achievement of ≤1% BCR-ABL (IS).

As used herein, “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable carrier” refers to an excipient or vehicle that includes without limitation diluents, disintegrants, precipitation inhibitors, surfactants, glidants, binders, lubricants, and the like with which the compound is administered. Carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The term “pharmaceutically acceptable salt” or “therapeutically acceptable salt” refer to a salt form of a compound of Formula (I) which is, within the scope of sound medical evaluation, suitable for use in contact with the tissues and organs of humans and/or animals such that any resulting toxicity, irritation, allergic response, and the like and are commensurate with a reasonable benefit/risk ratio. “Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ-COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

Physiologically acceptable salts (e.g. pharmaceutically acceptable salt) of the compounds of the invention include salts derived from an appropriate base, such as an alkali metal or an alkaline earth (for example, Na⁺, Li⁺, K+, Ca⁺² and Mg⁺²), ammonium and NR₄. Physiologically acceptable salts of a nitrogen atom or an amino group include (a) acid addition salts formed with inorganic acids, for example, aspartic acid, hydrochloric acid, hydrobromic acid, sulfuric acid, sulfamic acids, phosphoric acid, nitric acid and the like; (b) salts formed with organic acids such as, for example, acetic acid, trifluoroacetic acid, oxalic acid, tartaric acid, succinic acid, maleic acid, fumaric acid, gluconic acid, citric acid, malic acid, ascorbic acid, benzoic acid, 4-acetamido-benzoic acid, caproic acid, caprylic acid, adipic acid, isethionic acid, lactobionic acid, tannic acid, palmitic acid, alginic acid, glutamic acid, polyglutamic acid, formic acid, naphthalenesulfonic acid, naphthalene-2-sulfonic acid, naphthalene-1,5-disulfonic acid, methanesulfonic acid, cyclamic acid, p-toluenesulfonic acid, benzenesulfonic acid, lauryl sulfuric acid, polygalacturonic acid, malonic acid, nicotinic acid, galacturonic acid, pyruvic acid, camphoric acid, cinnamic acid, sulfosalicylic acid, glycolic acid, 2-hydroxy-3-naphthoate, 2-hydroxy ethane sulfonic acid, pamoate, salicylic acid, stearic acid, phthalic acid, mandelic acid, melonic acid, hippuric acid, lactic acid, ethanesulfonic acid, lysine, arginine, glycine, serine, threonine, alanine, isoleucine, leucine and the like; and (c) salts formed from elemental anions for example, chlorine, bromine, and iodine. Physiologically acceptable salts of a compound of a hydroxy group include the anion of said compound in combination with a suitable cation such as Na⁺ and NR₄₊. Each R is independently selected from H and (C₁-C₈) alkyl.

The term “level of expression” herein refers to the rate of processing information from a gene (such as a BCR-ABL with or without T315I, F317L, E255K and Y253F mutations) in the synthesis of a gene product, particularly of a functional gene product. In different embodiments, gene expression may be indicated by transcriptional expression of mRNA levels or protein levels.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula I, and a guest (or co-former) molecule or molecules. In particular embodiments the pharmaceutically acceptable co-crystal of the compound of Formula I or of the compound of Formula II with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

### Methods of Treatment

Provided is a method of inhibiting the proliferation of cancer cells in a subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Provided herein is a method of inhibiting the proliferation of BCR-ABL-expressing cells in a subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Provided herein is a method of treatment of chronic myeloid leukemia (CML) in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Provided herein is a method of treatment of chronic phase chronic myeloid leukemia (CML) in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Provided herein is a method of treatment of accelerated phase chronic myeloid leukemia in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Provided herein is a method of treatment of blast phase chronic myeloid leukemia (CML) in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

For each of the types or phases of CML listed above, there is a separate method of treatment for the CML in question comprising administering to the subject in need thereof:
a) a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a therapeutically effective amount of a tyrosine kinase inhibitor selected from the group of imatinib (GLEEVEC®), nilotinib (TASIGNA®), dasatinib (SPRYCEL®), ponatinib (ICLUSIG®), and bosutinib (BOSULIF®),

Also provided herein is a method of treatment of acute lymphoblastic leukemia (ALL) in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of acute lymphocytic leukemia in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of lymphoma in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of solid tumors in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Additionally provided herein is a method of treatment of Ph-positive acute lymphoblastic leukemia (Ph+ ALL or Philadelphia chromosome-positive ALL) in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Further provided herein is a method of treatment of Ph-positive acute lymphoblastic leukemia (Ph+ ALL or Philadelphia chromosome-positive ALL) in a human subject, the method comprising administering to the subject in need thereof:
a) a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a therapeutically effective amount of at least one tyrosine kinase inhibitor selected from the group of imatinib (GLEEVEC®), dasatinib (SPRYCEL®), ponatinib (ICLUSIG®), bosutinib (BOSULIF®), asciminib (SCEMBLIX®), and nilotinib (TASIGNA®), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug of the one or more tyrosine kinase inhibitor(s).

Also provided herein is a method of treatment of Ph-positive acute lymphoblastic leukemia (Ph+ ALL or Philadelphia chromosome-positive ALL) in a human subject, the method comprising administering to the subject in need thereof:
a) a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a therapeutically effective amount of blinatumomab.

Also provided herein is a method of treatment of acute myelogenous leukemia (AML) in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of a myelodysplastic syndrome in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of gastric cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of endometrial cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of bladder cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of multiple myeloma in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of breast cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of prostate cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of lung cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof. A further embodiment provides such a method of treatment of lung cancer, wherein the lung cancer is non-small cell lung cancer.

Also provided herein is a method of treatment of colorectal cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of renal cancer in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided herein is a method of treatment of glioblastoma in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Further provided is a method of treatment of gastrointestinal stromal tumor in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

Also provided is the use of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in the preparation of a medicament for use as a pan-BCR-ABL inhibitor.

Reference herein to a disease or condition being associated with a BCR-ABL mutation indicates a BCR-ABL mutation in the kinase domain has been identified or is identifiable in a human subject of interest, including those in need of treatment for the disease or condition in question. Such a mutation may be associated with a disease or condition described herein, including, but not limited to CML, AML, and/or ALL. In some embodiments, the subject experiencing the BCR-ABL mutation has proven refractory or resistant to treatment with one or more pharmaceutical agents, such as a tyrosine kinase inhibitor (ponatinib, imatinib, dasatinib, nilotinib, etc.). It is understood that such a mutation may be referred to as the subject experiencing such a mutation, the subject having the mutation, or the disease or condition being associated with such a mutation, or a particular tumor having or expressing the product of such a mutation.

The BCR-ABL inhibiting compounds described herein may be produced by methods known in the art, including those in U.S. Pat. No. 8,859,553 (Yu et al.); US 2021/0393628 A1 (Mackall et al.); US 2015/0105377 (Gozgit et al.); Designing Novel BCR-ABL Inhibitors for Chronic Myeloid Leukemia with Improved Cardiac Safety, Pandrala et al., Journal of Medicinal Chemistry, 2022, 65, 10898-10919; Rapid Discovery of a Novel Series ofAbl Kinase Inhibitors byApplication of an Integrated Microfluidic Synthesis and Screening, Desai et al., Journal of Medicinal Chemistry, 2013, 56, 3033-3047; Discovery of 3-[2-(Imidazo[1,2-b]pyridazin-3-yl)ethynyl]-4-methyl-N-{4-[(4-methylpiperazin-1-yl)-methyl]-3-(trifluoromethyl)phenyl}benzamide (AP24534), a Potent, Orally Active Pan-Inhibitor of Breakpoint Cluster Region-Abelson (BCR-ABL)7 Kinase Including the T315I Gatekeeper Mutant, Huang et al., Journal of Medicinal Chemistry, 2010, 53, 4701-4719; Design, Synthesis, and Biological Evaluation of 3-(1H-1,2,3-Triazol-1-yl)benzamide Derivatives as Potent Pan Bcr-Abl Inhibitors Including the Threonine³¹⁵→Isoleucine³¹⁵ Mutant, Li et al., Journal of Medicinal Chemistry, 2012, 55, 10033-10046; and Design, Synthesis, and Biological Evaluation of 3-(Imidazo[1,2-a]pyrazin-3-ylethynyl)-4-isopropyl-N-(3-((4-methyl-piperazin-1-yl)methyl)-5-(trifluoromethyl)phenyl)benzamide as a Dual Inhibitor of Discoidin Domain Receptors 1 and 2, Wang et al., Journal of Medicinal Chemistry, 2018, 61, 7977-7990.

As a non-limiting example using methods from Pandrala et al., Journal of Medicinal Chemistry, 2022, 65, 10898-10919, 3-iodo-4-methylbenzoic acid (A) is reacted with 3-bromo-5-(trifluoromethyl)aniline (B) (SOCl₂, diisopropylethylamine, DMAP, THF, reflux, 5 hours, THF) to produce N-(3-bromo-5-(trifluoromethyl)phenyl)-3-iodo-4-methylbenzamide (C). C is then reacted with 3-ethynylimidazo[1,2-b]pyridazine (D) in the presence of Cul, [Pd(Ph₃P)₄], and DMF in a sealed tube at 100° C. for five hours to produce N-(3-bromo-5-(trifluoromethyl)phenyl)-3-(imidazo[1,2-b]pyridazin-3-ylethynyl)-4-methylbenzamide (E). Copper-catalyzed N-arylation can then be used to introduce the R2 group, particularly including 1-H-imidazole (CET-10-084), 2-methyl-1H-imidazole (CET-10-067), 4-fluoro-1H-imidazole (CET-10-100), 4-(trifluoromethyl)-1H-imidazole (CET-10-103), and 4-methyl-1H-imidazole (CET-10-108).

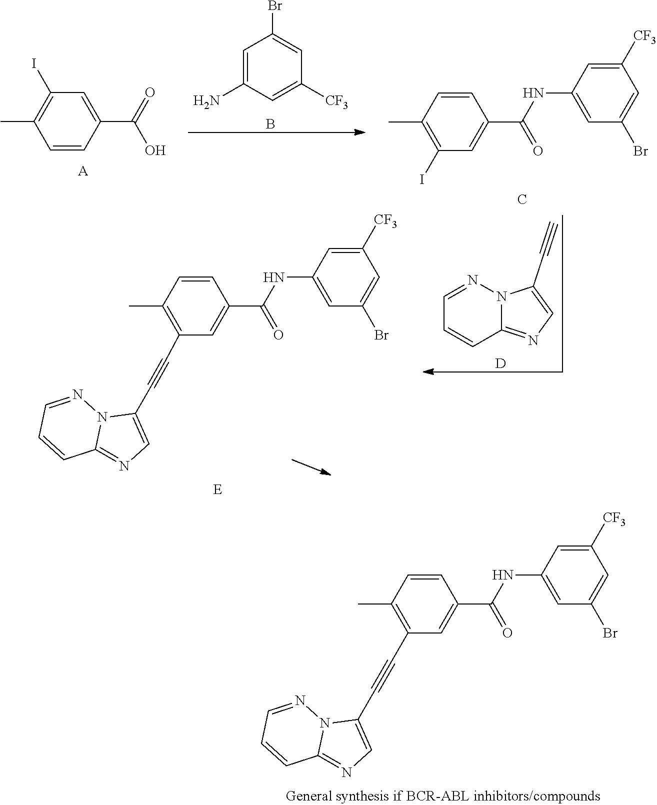

### Synthesis of Coupled Aryl Aniline

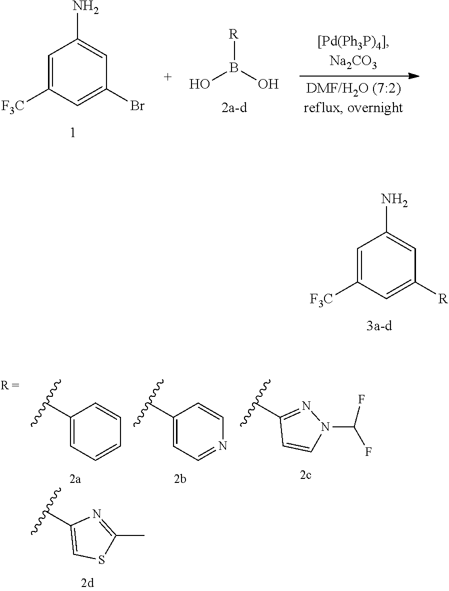

### Synthesis of the HIT Compound

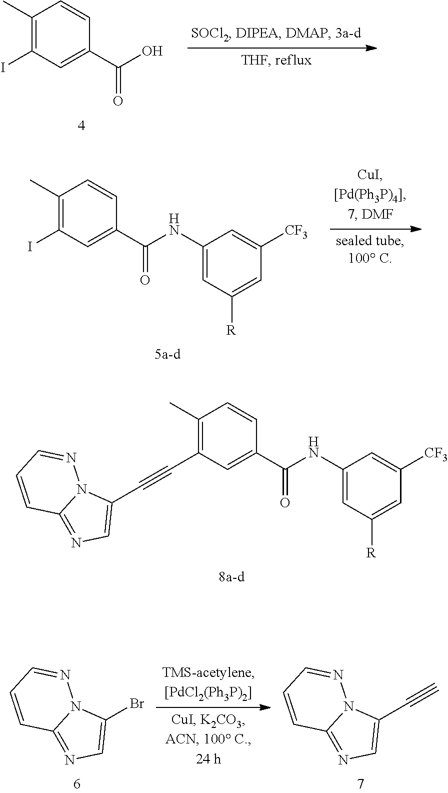

The General procedure for the synthesis of coupled aryl amine.

The desired aryl amines were synthesized by reported procedure.¹ The 3-bromo-5-(trifluoromethyl)aniline 1 (1 mmol, 1 equiv) with corresponding aryl boronic acid 2a-d (2 mmol, 2 equiv), tetrakis(triphenylphosphine)palladium(0) (0.025 mmol, 0.025 equiv), and Na₂CO₃ (7 mmol, 7 equiv) were combined in 30 mL deoxygenated DMF/H₂O (7:2) and heated to reflux overnight. The reaction was then cooled to room temperature and diluted with DCM/hexanes (1:1). The combined organic layers were washed with saturated aqueous NaHCO₃ (2×25 mL), water (2×25 mL), and brine (25 mL), dried over Na₂SO₄, filtered, and concentrated under reduced pressure. The crude residue was purified by flash column chromatography (SiO2, 30%→80% EtOAc/hexane) to afford desired coupled aryl aniline 3a-d.

### Synthesis of Compound 5a-d

Under a nitrogen atmosphere, 3-iodo-4-methylbenzoic acid 4 (20 mmol, 1 equiv) was taken in SOCl₂ (100 mmol, 5 equiv) and then two drops of DMF was added at rt. The reaction mixture was stirred at reflux for 5 h before it was cooled to rt, and the excess SOCl₂ was carefully removed. The crude material was co-evaporated with benzene and dried under vacuum to afford the desired acid chloride. The acid chloride was dissolved in anhydrous THE (20 mL) and then added dropwise to a stirred mixture of 3-bromo-5-(trifluoromethyl)aniline 3a-d (20 mmol, 1 equiv), diisopropylethylamine (24 mmol, 1.2 equiv), and DMAP (2.0 mmol, 0.1 equiv) in THE at 0° C. Upon completion of the addition, the reaction mixture was warmed to rt and stirred overnight. The reaction was quenched with water, and the product was extracted into EtOAc (3×50 mL). The combined organic extracts were washed with a brine solution (25 mL), dried over Na₂SO₄, filtered, and evaporated to dryness to afford a crude material that was purified on a silica gel column using a 0% to 50% gradient of EtOAc in hexane as the eluent to obtain the desired product 5a-d as an off-white solid.

### Synthesis of Alkyne 7

Compound 7 was prepared according to the previously reported method,² with several modifications. To a solution of 3-bromoimidazo[1,2-b]pyridazine 6 (10.0 g, 50.5 mmol) in acetonitrile were added CuI (0.5 g, 2.63 mmol), Pd(PPh₃)₂C₁₂ (1.8 g, 2.63 mmol), and TEA (21.0 mL, 150.6 mmol). The solution was purged with a nitrogen flow for 10 min, and then ethynyltrimethylsilane (21.0 mL, 151.8 mmol) was added. The mixture was heated to reflux overnight. After being cooled to rt, the reaction mixture was filtered to remove the undissolved solid. The solid was washed with copious amounts of acetonitrile. The filtrate was evaporated to dryness and then placed in methanol (300 mL). To this mixture was added K₂CO₃ (14.3 g, 103.5 mmol) at room temperature, and then the mixture was stirred for 4 h. The progress of the reaction was monitored by TLC. The reaction mixture was filtered to remove excess K₂CO₃. The solid was washed with a minimal amount of methanol. The filtrate was concentrated to dryness, dissolved in excess EtOAc, and then washed with water followed by a brine solution. The organic phase was dried over Na2SO4, filtered, and evaporated to dryness to afford the crude product, which was purified on a silica gel column using a 0% to 50% gradient of EtOAc in hexane to afford the desired product as a pale-brown solid (5.0 g, 69%).

### Synthesis of Compound 8a-d

Compound 8a-d was prepared according to the literature procedure,³ with few modifications. 3-Iodo-4-methylbenzoate derivative 5a-d (6.71 mmol, 1.2 equiv) was added to a stirred solution of 3-ethynylimidazo[1,2-b]pyridazine 7 (5.59 mmol, 1 equiv) in DMF (10 mL). The mixture underwent three cycles of vacuum/filling with nitrogen, and then CuI (1.11 mmol, 0.2 equiv), Pd(PPh₃)₄ (0.55 mmol, 0.1 equiv), and diisopropylethylamine (11.17 mmol, 2 equiv) were added. The reaction mixture was stirred at 80° C. for 2 h before it was cooled to rt. Water (25 mL) was added, and the product extracted into EtOAc (3×25 mL). The organic layers were combined and washed with water (20 mL) followed by a brine solution (20 mL). The organic phase was dried over Na₂SO₄, filtered, and then evaporated to dryness to afford a gummy solid, which was then triturated with minimal acetonitrile to yield a solid. The solid was collected by filtration, washed with a minimal amount of acetonitrile, and dried under vacuum for 2 h to furnish the desired compound 8a-d as an off-white solid.

### REFERENCES

1. V. L. Gunderson, A. L. Smeigh, C. H. Kim, D. T. Co, M. R. Wasielewski, J.Am.Chem.Soc.2012, 134, 4363-4372.
2. W. S. Huang, C. A. Metcalf, R. Sundaramoorthi, Y. Wang, D. Zou, R. M. Thomas, X. Zhu, L. Cai, D. Wen, S. Liu, J. Romero, J. Qi, I. Chen, G. Banda, S. P. Lentini, S. Das, Q. Xu, J. Keats, F. Wang, S. Wardwell, Y. Ning, J. T. Snodgrass, M. I. Broudy, K. Russian, T. Zhou, L. Commodore, N. I. Narasimhan, Q. K. Mohemmad, J. luliucci, V. M. Rivera, D. C. Dalgarno, T. K. Sawyer, T. Clackson, W. C. Shakespeare, J. Med. Chem. 2010, 53, 4701-4719.
3. M. Najjar, C. Suebsuwong, S. S. Ray, R. J. Thapa, J. L. Maki, S. Nogusa, S. Shah, D. Saleh, P. J. Gough, J. Bertin, et al. Cell Rep. 2015, 10, 1850-1860.

The following specific compounds have been made and tested and provide a set of non-limiting species falling within Formula (I) and other formulas provided herein.

### Example No. 1—N-(3-(1H-imidazol-1-yl)-5-(trifluoromethyl)phenyl)-3-(imidazo[1,2-b]pyridazin-3-ylethynyl)-4-methylbenzamide (CET-10-084)

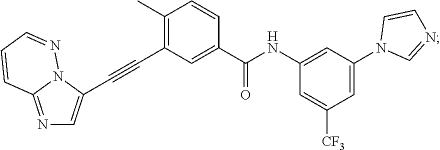 [SMILES: Cc1ccc(C(=O)Nc2cc(-n3ccnc3)cc(C(F)(F)F)c2)cc1C#Cc1cnc2cccnn12]

### Example No. 2—3-(imidazo[1,2-b]pyridazin-3-ylethynyl)-4-methyl-N-(3-(2-methyl-1H-imidazol-1-yl)-5-(trifluoromethyl)phenyl)benzamide (CET-10-067)

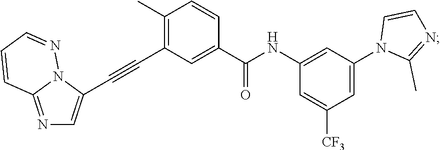 [SMILES: Cc1ccc(C(=O)Nc2cc(-n3ccnc3C)cc(C(F)(F)F)c2)cc1C#Cc1cnc2cccnn12]

HRMS (ESI-TOF) m/z of Compound CET-10-067: [M+Na]⁺Calcd for C₂₇H₁₉F₃N₆ONa is 523.1470.

### Example No. 3—N-(3-(4-fluoro-1H-imidazol-1-yl)-5-(trifluoromethyl)phenyl)-3-(imidazo[1,2-b]pyridazin-3-ylethynyl)-4-methylbenzamide (CET-10-100)

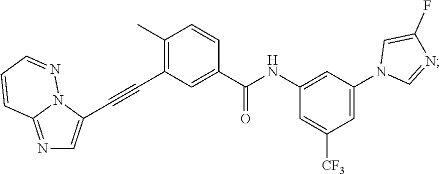 [SMILES: Cc1ccc(C(=O)Nc2cc(-n3cnc(F)c3)cc(C(F)(F)F)c2)cc1C#Cc1cnc2cccnn12]

HRMS (ESI-TOF) m/z of Compound CET-10-100: [M+Na]⁺Calcd for C₂₆H₁₆F₄N₆ONa is 523.1470;

### Example No. 4—3-(imidazo[1,2-b]pyridazin-3-ylethynyl)-4-methyl-N-(3-(trifluoromethyl)-5-(4-(trifluoromethyl)-1H-imidazol-1-yl)phenyl)benzamide (CET-10-103)

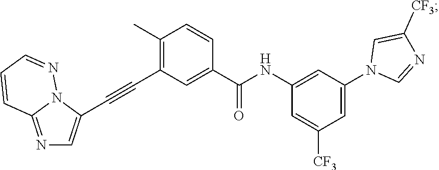 [SMILES: Cc1ccc(C(=O)Nc2cc(-n3cnc(C(F)(F)F)c3)cc(C(F)(F)F)c2)cc1C#Cc1cnc2cccnn12]

HRMS (ESI-TOF) m/z of Compound CET-10-103: [M+Na]⁺Calcd for C₂₇H₁₆F₆N₆ONa is 577.1187;

### Example No. 5—3-(imidazo[1,2-b]pyridazin-3-ylethynyl)-4-methyl-N-(5-(trifluoromethyl)-[1,1′-biphenyl]-3-yl)benzamide (CET-10-106)

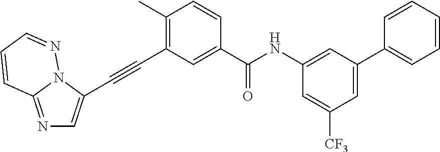 [SMILES: Cc1ccc(C(=O)Nc2cc(-c3ccccc3)cc(C(F)(F)F)c2)cc1C#Cc1cnc2cccnn12]

HRMS (ESI-TOF) m/z of Compound CET-10-106: [M+Na]⁺Calcd for C₂₉H₁₉F₃N₄0Na is 519.1408;

### Example No. 6—3-(imidazo[1,2-b]pyridazin-3-ylethynyl)-4-methyl-N-(3-(pyridin-4-yl)-5-(trifluoromethyl) phenyl)benzamide (CET-10-104)

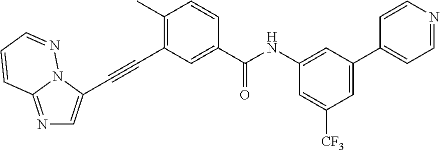 [SMILES: Cc1ccc(C(=O)Nc2cc(-c3ccncc3)cc(C(F)(F)F)c2)cc1C#Cc1cnc2cccnn12]

HRMS (ESI-TOF) m/z of Compound CET-10-104: [M+Na]⁺Calcd for C₂₈H₁₈F₃N₅ONa is 520.1361;

### Example No. 7—N-(4-(1H-imidazol-1-yl)-3-(trifluoromethyl)phenyl)-3-(imidazo[1,2-b]pyridazin-3-ylethynyl)-4-methylbenzamide (CET-10-105)

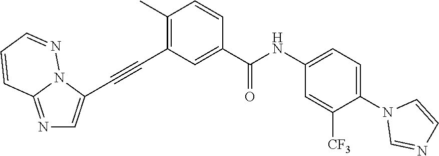 [SMILES: Cc1ccc(C(=O)Nc2ccc(-n3ccnc3)c(C(F)(F)F)c2)cc1C#Cc1cnc2cccnn12]

HRMS (ESI-TOF) m/z of Compound CET-10-105: [M+Na]⁺Calcd for C₂₆H₁₇F₃N₆ONa is 509.1313;

### Example No. 8—N-(3-(1-(difluoromethyl)-1H-pyrazol-4-yl)-5-(trifluoromethyl)phenyl)-3-(imidazo[1,2-b]pyridazin-3-ylethynyl)-4-methylbenzamide (CET-10-109)

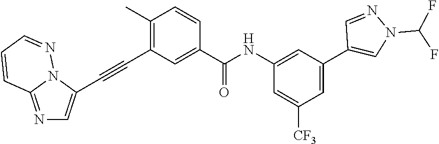 [SMILES: Cc1ccc(C(=O)Nc2cc(-c3cnn(C(F)F)c3)cc(C(F)(F)F)c2)cc1C#Cc1cnc2cccnn12]

HRMS (ESI-TOF) m/z of Compound CET-10-109: [M+Na]⁺Calcd for C₂₇H₁₇F₅N₆ONa is 559.1281;

### Example No. 9—3-(imidazo[1,2-b]pyridazin-3-ylethynyl)-4-methyl-N-(3-(methylsulfonamido)-5-(trifluoromethyl)phenyl)benzamide (CET-10-110)

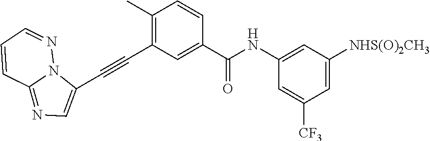

HRMS (ESI-TOF) m/z of Compound CET-10-110: [M+Na]⁺Calcd for C₂₄H₁₈F₃N₅O₃SNa is 536.0980;

### Example No. 10—3-(imidazo[1,2-b]pyridazin-3-ylethynyl)-4-methyl-N-(3-(2-methylthiazol-4-yl)-5-(trifluoromethyl)phenyl)benzamide (CET-10-111)

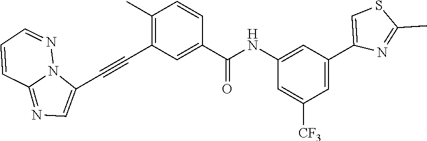 [SMILES: Cc1nc(-c2cc(NC(=O)c3ccc(C)c(C#Cc4cnc5cccnn45)c3)cc(C(F)(F)F)c2)cs1]

HRMS (ESI-TOF) m/z of Compound CET-10-111: [M+Na]⁺Calcd for C₂₇H₁₃F₃N₅OSNa is 540.1081;

### Example No. 11—3-(2-(imidazo[1,2-b]pyridazin-3-yl)vinyl)-4-methyl-N-(5-(trifluoromethyl)-[1,1′-biphenyl]-3-yl)benzamide (CET-10-101)

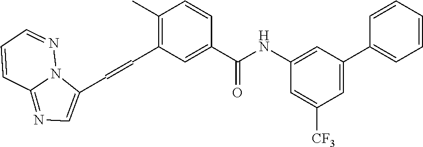 [SMILES: Cc1ccc(C(=O)Nc2cc(-c3ccccc3)cc(C(F)(F)F)c2)cc1C=Cc1cnc2cccnn12]

HRMS (ESI-TOF) m/z of Compound CET-10-101: [M+Na]⁺Calcd for C₂₉H_{d 1}F₃N₄0Na is 521.1565;

### Example No. 12—3-(2-(imidazo[1,2-b]pyridazin-3-yl)vinyl)-4-methyl-N-(3-(pyridin-4-yl)-5-(trifluoromethyl)phenyl)benzamide (CET-10-102)

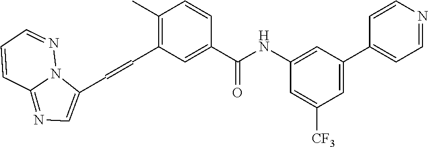 [SMILES: Cc1ccc(C(=O)Nc2cc(-c3ccncc3)cc(C(F)(F)F)c2)cc1C=Cc1cnc2cccnn12]

HRMS (ESI-TOF) m/z of Compound CET-10-102: [M+Na]⁺Calcd for C₂₈H₂₀F₃N₅ONa is 522.1517;

### Example No. 13—3-(4-(imidazo[1,2-b]pyridazin-3-yl)-1H-1,2,3-triazol-1-yl)-4-methyl-N-(3-(4-methyl-1H-imidazol-1-yl)-5-(trifluoromethyl)phenyl)benzamide (CET-10-108)

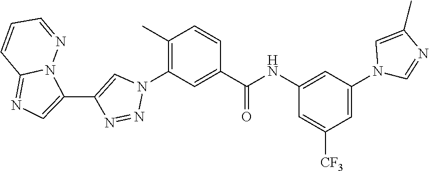 [SMILES: Cc1cn(-c2cc(NC(=O)c3ccc(C)c(-n4cc(-c5cnc6cccnn56)nn4)c3)cc(C(F)(F)F)c2)cn1]

HRMS (ESI-TOF) m/z of Compound CET-10-108: [M+Na]⁺Calcd for C₂₇H₂₀F₃N₉ONa is 566.1640;

### Example No. 14—N-(3-(1H-imidazol-1-yl)-5-(trifluoromethyl)phenyl)-3-(4-(imidazo[1,2-b]pyridazin-3-yl)-1H-1,2,3-triazol-1-yl)-4-methylbenzamide (CET-10-107)

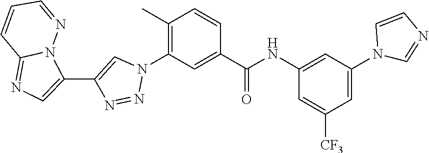 [SMILES: Cc1ccc(C(=O)Nc2cc(-n3ccnc3)cc(C(F)(F)F)c2)cc1-n1cc(-c2cnc3cccnn23)nn1]

HRMS (ESI-TOF) m/z of Compound CET-10-107: [M+Na]⁺Calcd for C₂₆H₁₃F₃N₉ONa is 552.1484; or a pharmaceutically acceptable salt, co-crystal, ester, solvate, hydrate, isomer (including optical isomers, rafcemates, or other mixtures thereof), tautomer, isotope, polymorph, or pharmaceutically acceptable prodrug thereof.

### Cell Proliferation Assay

A cell proliferation assay using murine pro-B cell lines (Ba/F₃) stably expressing native or mutated BCR-ABL. Ba/F₃ cells are interleukin-3 (IL3) dependent. However, expression of BCR-ABL makes them IL3 independent and able to grow without IL3 signaling. Therefore, BCR-ABL inhibition results in a detectable reduction of cell proliferation using a tetrazolium inner salt (MTS) and reveals the pharmacodynamic potency of candidate TKIs.

For this project we were desiring a TKI who had a pharmacodynamic profile similar to Ponatinib when it came to potency in this assay. We also used cells that did not express BCR-ABL to show less toxicity through being specific to just this target potentially. Imatinib was our negative drug control, and ponatinib our positive drug control.

K562 cells as well as Ba/F₃ parental cells or those transduced with empty vector or vectors expressing BCR-ABL WT or a series of point mutations known to confer resistance to ABL tyrosine kinase inhibitors were seeded into 384-well plates at 750 cells per well in RPMI media containing 10% FBS and supplemented with L-Glutamine, Penicillin/Streptomycin, and fungizone (amphotericin B). Cells were incubated with vehicle or graded concentrations of indicated inhibitors in triplicate for 72 hours at which time a tetrazolium-based MTS assay was used to quantify relative numbers of viable cells. After incubating the cells for 1-4 hours with the MTS reagent, absorbance values were read at 490 nm using a Synergy 2 plate reader. The absorbance values from blank wells containing media alone were subtracted from all other wells and then absorbance values from all wells were normalized to the average of replicate vehicle controls to produce normalized percent viability values. These normalized values were curve fit to generate dose response metrics for each inhibitor.

What is claimed:

1. A compound of Formula (I): 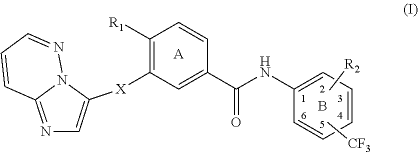
wherein:
X is selected from the group of: 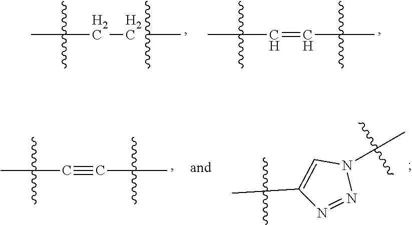
R₁ is selected from the group of C₁-C₆ alkyl, C₁-C₆ alkoxy, C₃-C₆ cycloalkyl, —CH₂—C₃-C₆ cycloalkyl, —O—C₃-C₆ cycloalkyl, halogen, C₁-C₃ haloalkyl, OH, and CN;
R₂ is selected from the group of H, —NH—S(═O)₂H, —NH—S(═O)₂—C₁-C₆ alkyl, 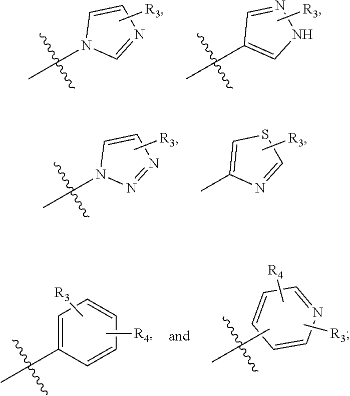
R₃ and R₄ are each independently selected from the group of H, C₁-C₆ alkyl, —O—C—C₆ alkyl, C₁-C₆ haloalkyl, halogen, —NH—S(═O)₂H, and —NH—S(═O)₂—C₁-C₆ alkyl;
with the proviso that, when X is 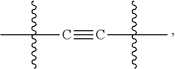 [SMILES: *C#C*]
R₁ is CH₃ or Cl, and the variable CF₃ group is bound to either position 3 or position 5 of phenyl ring B, then R₂ is not 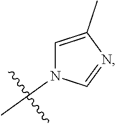 [SMILES: *n1cnc(C)c1]
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

2. The compound of claim 1, having Formula (Ia): 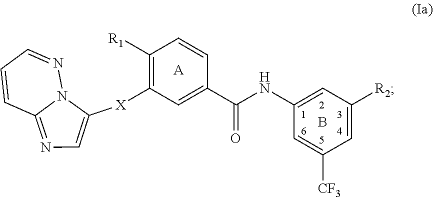
wherein X, R₁ and R₂ are as defined in claim 1, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

3. The compound of claim 1, having Formula (Ib): 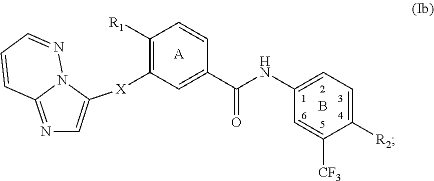
wherein X, R₁ and R₂ are as defined in claim 1, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

4. The compound of claim 1, having Formula (Ic): 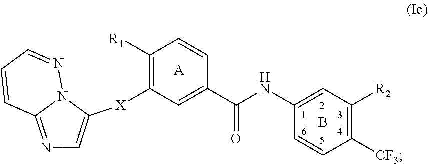
wherein X, R₁ and R₂ are as defined in claim 1, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

5. The compound of claim 1, having Formula (IIa): 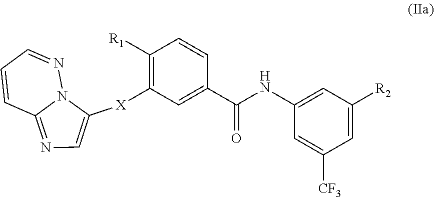
wherein: X is selected from the group of: 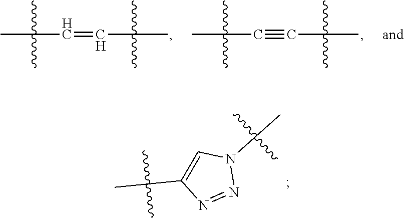
R₁ is selected from the group of C₁-C₄ alkyl, C—C₄ alkoxy, C₃-C₆ cycloalkyl, —CH₂—C₃-C₆ cycloalkyl, —O—C₃-C₆ cycloalkyl, halogen, C₁-C₃ haloalkyl, OH, and CN;
R₂ is selected from the group of H, —NH—S(═O)₂H, —NH—S(═O)₂—C₁-C₆ alkyl, 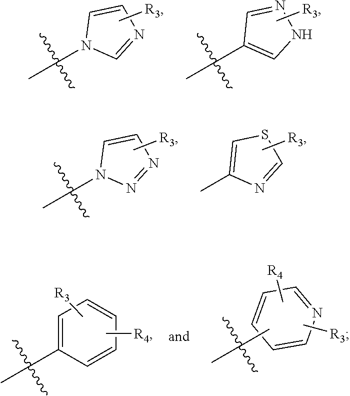
R₃ and R₄ are each independently selected from the group of H, C₁-C₆ alkyl, C—C₆ haloalkyl, halogen, —NH—S(═O)₂H, and —NH—S(═O)₂—C₁-C₆ alkyl;
with the proviso that, when X is 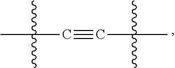 [SMILES: *C#C*]
R₁ is CH₃ or Cl, and the variable CF₃ group is bound to either position 3 or position 5 of phenyl ring B, then R₂ is not 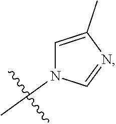 [SMILES: *n1cnc(C)c1]
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

6. The compound of claim 1, having Formula (IIb): 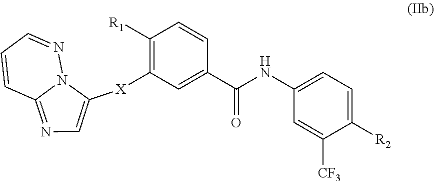
wherein X, R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

7. The compound of claim 1, having Formula (IIc): 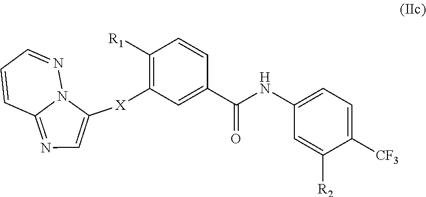
wherein X, R₁ and R2 are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

8. The compound of claim 1, having Formula (IIIa): 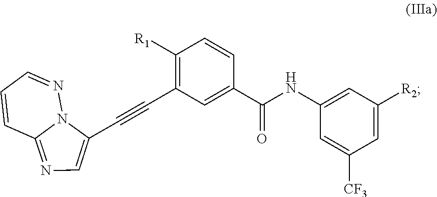
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

9. The compound of claim 1, having Formula (IIIb): 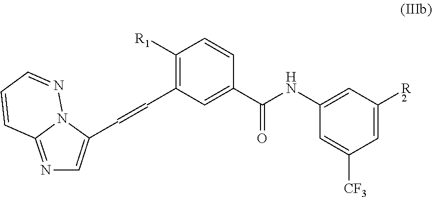
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

10. The compound of claim 1, having Formula (IIIc): 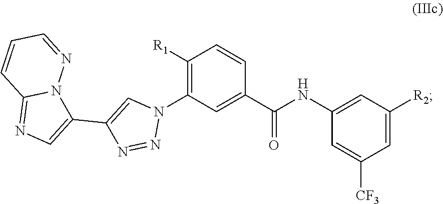
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

11. The compound of claim 1, having Formula (IIId): 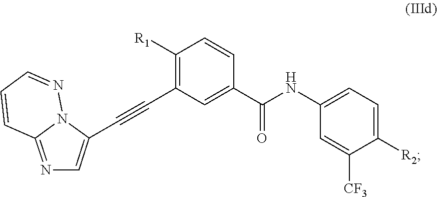
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

12. The compound of claim 1, having Formula (IIIe): 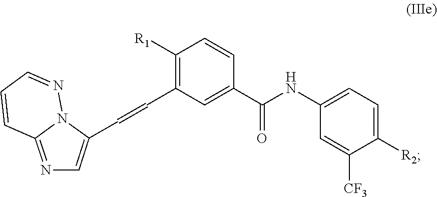
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

13. The compound of claim 1, having Formula (IIIf): 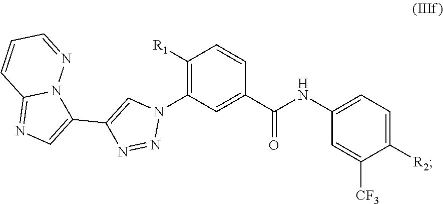
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

14. The compound of claim 1, having Formula (IIIg): 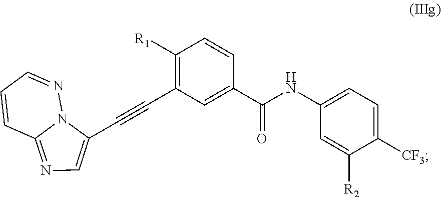
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

15. The compound of claim 1, having Formula (IIIh): 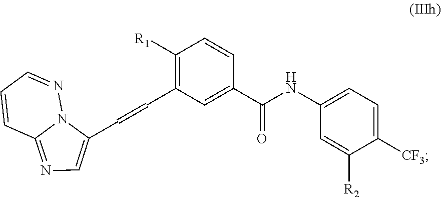
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

16. The compound of claim 1, having Formula (IIIi): 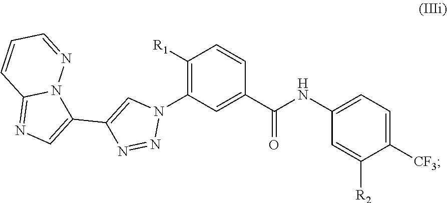
wherein R₁ and R₂ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

17. The compound of claim 1, having Formula (IVa): 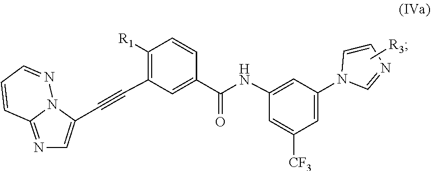
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

18. The compound of claim 1, having Formula (IVb): 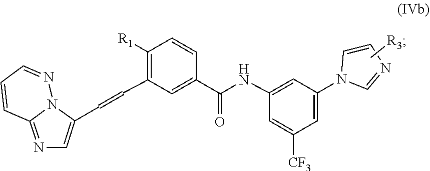
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

19. The compound of claim 1, having Formula (IVc): 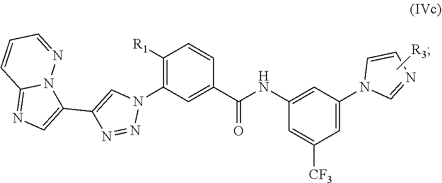
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

20. The compound of claim 1, having Formula (IVd): 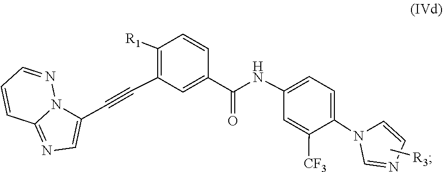
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

21. The compound of claim 1, having Formula (IVe): 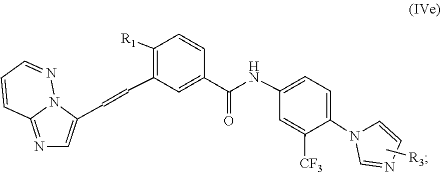
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

22. The compound of claim 1, having Formula (IVf): 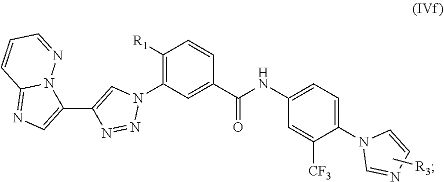
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

23. The compound of claim 1, having Formula (IVg): 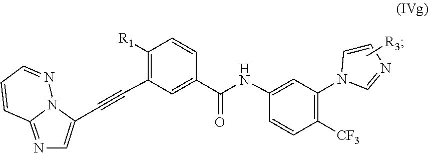
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

24. The compound of claim 1, having Formula (IVh): 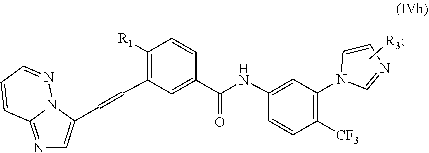
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

25. The compound of claim 1, having Formula (IVi): 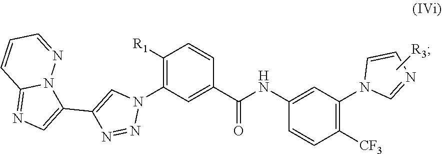
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

26. The compound of claim 1, having Formula (Va): 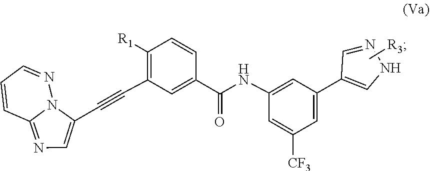
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

27. The compound of claim 1, having Formula (Vb): 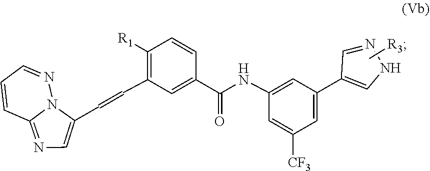
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

28. The compound of claim 1, having Formula (Vc): 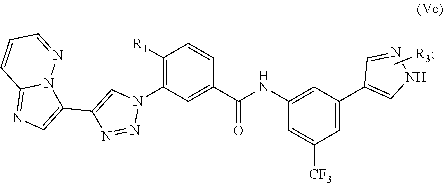
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

29. The compound of claim 1, having Formula (Vd): 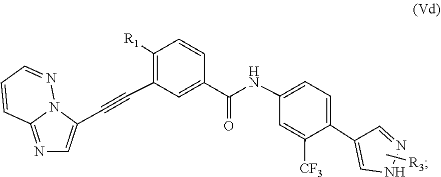
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

30. The compound of claim 1, having Formula (Ve): 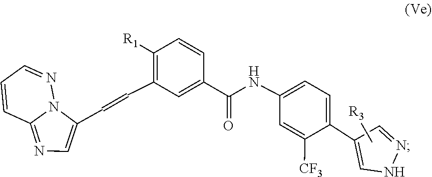
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

31. The compound of claim 1, having Formula (Vf): 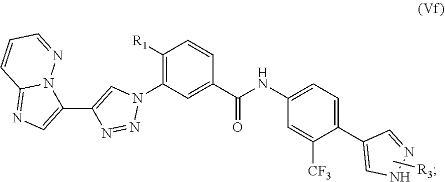
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

32. The compound of claim 1, having Formula (Vg): 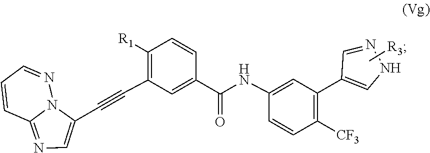
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

33. The compound of claim 1, having Formula (Vh): 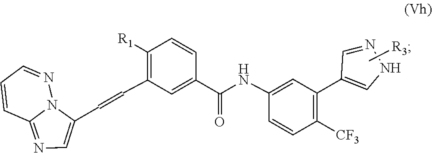
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

34. The compound of claim 1, having Formula (Vi): 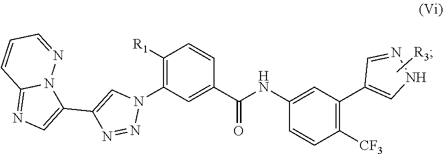
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

35. The compound of claim 1, having Formula (VIa): 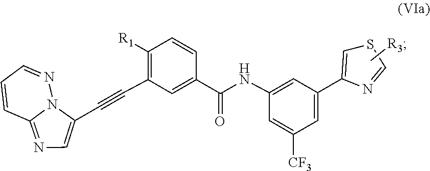
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

36. The compound of claim 1, having Formula (VIb): 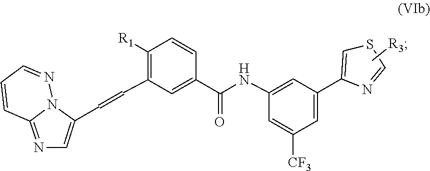
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

37. The compound of claim 1, having Formula (VIc): 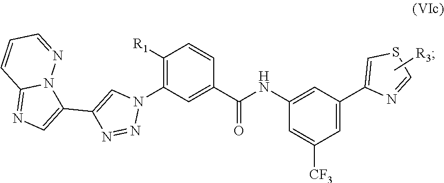
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

38. The compound of claim 1, having Formula (VId): 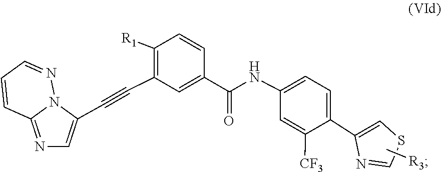
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

39. The compound of claim 1, having Formula (VIe): 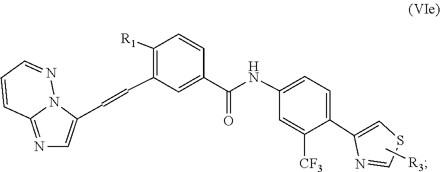
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

40. The compound of claim 1, having Formula (VIf): 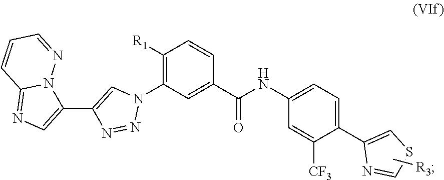
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

41. The compound of claim 1, having Formula (VIg): 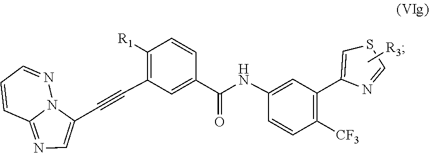
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

42. The compound of claim 1, having Formula (VIh): 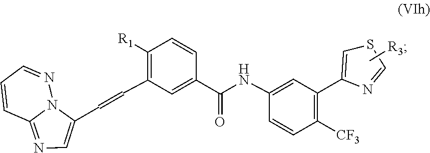
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

43. The compound of claim 1, having Formula (VIi): 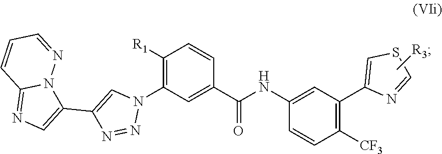
wherein R₁ and R₃ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

44. The compound of claim 1, having Formula (VIIa): 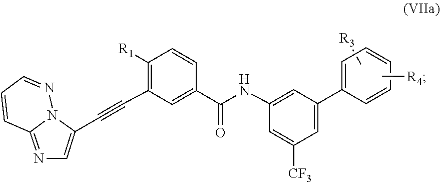
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

45. The compound of claim 1, having Formula (VIIb): 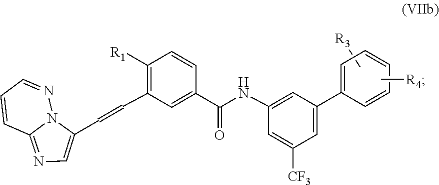
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

46. The compound of claim 1, having Formula (VIIc): 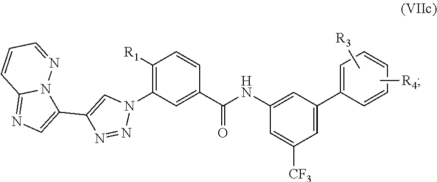
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

47. The compound of claim 1, having Formula (VIId): 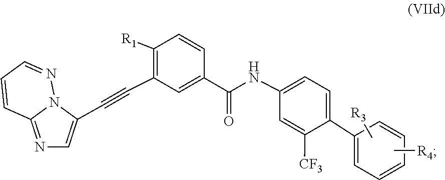
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

48. The compound of claim 1, having Formula (VIIe): 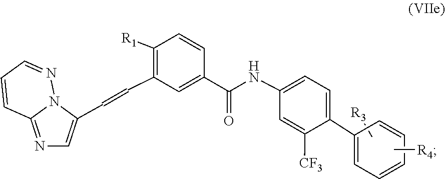
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

49. The compound of claim 1, having Formula (VIIf): 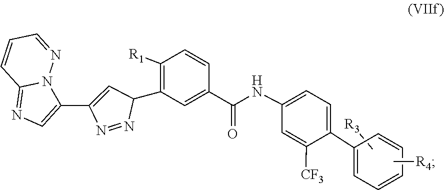
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

50. The compound of claim 1, having Formula (VIIg): 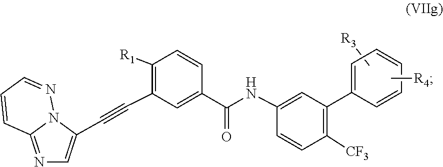
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

51. The compound of claim 1, having Formula (VIIh): 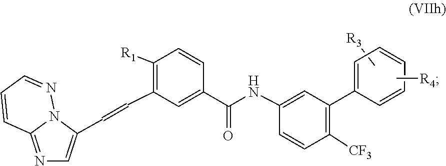
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

52. The compound of claim 1, having Formula (VIIi): 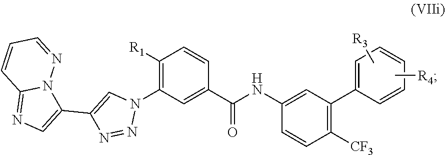
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

53. The compound of claim 1, having Formula (VIIIa): 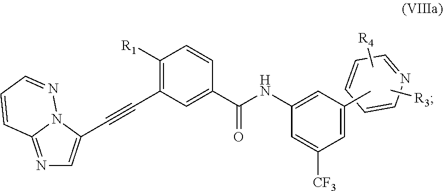
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

54. The compound of claim 1, having Formula (VIIIb): 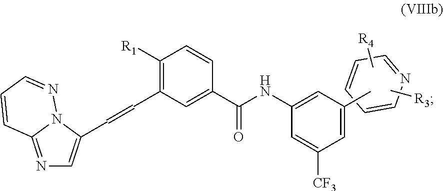
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

55. The compound of claim 1, having Formula (VIIIc): 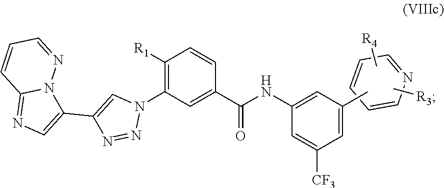
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

56. The compound of claim 1, having Formula (VIIId): 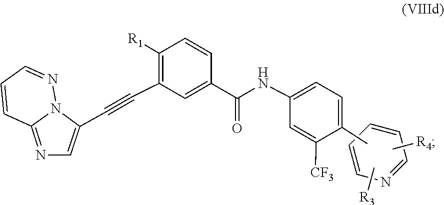
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

57. The compound of claim 1, having Formula (VIIIe): 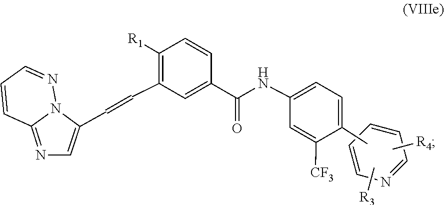
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

58. The compound of claim 1, having Formula (VIIIf): 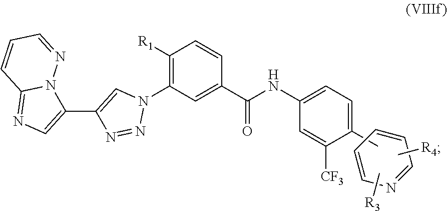
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

59. The compound of claim 1, having Formula (VIIIg): 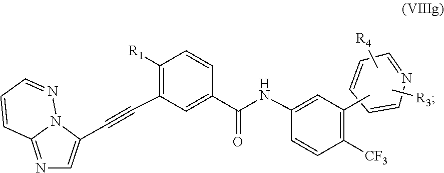
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

60. The compound of claim 1, having Formula (VIIIh): 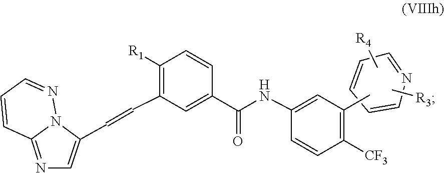
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

61. The compound of claim 1, having Formula (VIIIi): 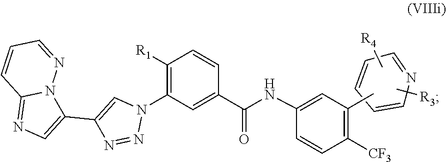
wherein R₁, R₃, and R₄ are as defined in claim 5, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

62. The compound of any of claims 59, 60, and 61, wherein the pyridine ring substituted by R₃ and R₄ is bound as a 4-pyridinyl ring; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

63. The compound of any of claims 59, 60, and 61, wherein the pyridine ring substituted by R₃ and R₄ is bound as a 3-pyridinyl ring; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

64. The compound of any of claims 59, 60, and 61, wherein the pyridine ring substituted by R₃ and R₄ is bound as a 2-pyridinyl ring; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

65. The compound of any of claims 1-64, wherein R₁ is selected from the group of C₁-C₄ alkyl, Cr—C₄ alkoxy, C₃-C₆ cycloalkyl, —CH₂—C₃-C₆ cycloalkyl, —O—C₃-C₆ cycloalkyl, halogen, C₁-C₃ haloalkyl, OH, and CN; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

66. The compound of any of claims 1-65, wherein R₁ is selected from the group of C₁-C₃ alkyl, C₁-C₃ alkoxy, C₃-C₆ cycloalkyl, —CH₂—C₃-C₆ cycloalkyl, —O—C₃-C₆ cycloalkyl, halogen, C₁-C₃ haloalkyl, OH, and CN; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

66. The compound of any of claims 1-65, wherein R₁ is selected from the group of C₁-C₃ alkyl, C₁-C₃ alkoxy, C₃-C₆ cycloalkyl, —CH₂-C₃-C₆ cycloalkyl, —O—C₃-C₆ cycloalkyl, halogen, and C₁-C₃ haloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

67. The compound of any of claims 1-66, wherein R₁ is selected from the group of C₁-C₃ alkyl, C₁-C₃ alkoxy, halogen, and C₁-C₃ haloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

68. The compound of any of claims 1-66, wherein R₁ is selected from the group of C₃-C₆ cycloalkyl, —CH₂—C₃-C₆ cycloalkyl, and —O—C₃-C₆ cycloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

69. The compound of any of claims 1-67, wherein R₁ is selected from the group of halogen and C₁-C₃ haloalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

70. The compound of any of claims 1-67, wherein R₁ is selected from the group of halogen and C₁-C₃ fluoroalkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

71. The compound of any of claims 1-67, wherein R₁ is selected from the group of C₁-C₃ alkyl and C₁-C₃ alkoxy; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

72. The compound of any of claims 1-67, wherein R₁ is C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

73. The compound of any of claims 1-67 and 72, wherein R₁ is —CH₃; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

73. The compound of any of claims 1-67 and 72, wherein R₁ is ethyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

74. The compound of any of claims 1-67 and 72, wherein R₁ is selected from the group of n-propyl and isopropyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

75. The compound of any of claims 1-74, wherein R₃ is selected from the group of H, C₁-C₄ alkyl, —O—C—C₄ alkyl, C—C₄ haloalkyl, halogen, —NH—S(═O)₂H, and —NH—S(═O)₂—C₁-C₄ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

76. The compound of any of claims 1-75, wherein R₃ is selected from the group of H, C₁-C₃ alkyl, —O—C₁-C₃ alkyl, C₁-C₃ haloalkyl, halogen, —NH—S(═O)₂H, and —NH—S(═O)₂—C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

77. The compound of any of claims 1-76, wherein R₃ is selected from the group of H, C₁-C₃ alkyl, and —O—C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

78. The compound of any of claims 1-76, wherein R₃ is selected from the group of C₁-C₃ haloalkyl and halogen; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

79. The compound of any of claims 1-76 and 78, wherein R3 is selected from the group of C₁-C₃ fluoroalkyl and F; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

80. The compound of any of claims 1-76, wherein R₃ is selected from the group of —NH-S(=O)₂H and —NH—S(═O)₂—C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

81. The compound of any of claims 1-76, wherein R₃ is H; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

82. The compound of any of claims 1-16 and 44-81, wherein R₄ is selected from the group of H, C₁-C₄ alkyl, —O—C—C₄ alkyl, C—C₄ haloalkyl, halogen, —NH—S(═O)₂H, and —NH—S(═O)₂—C₁-C₄ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

83. The compound of any of claims 1-16 and 44-81, wherein R₄ is selected from the group of H, C₁-C₃ alkyl, —O—C₁-C₃ alkyl, C₁-C₃ haloalkyl, halogen, —NH—S(═O)₂H, and —NH—S(═O)₂—C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

84. The compound of any of claims 1-16 and 44-81, wherein R₄ is selected from the group of H, C₁-C₃ alkyl, and —O—C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

85. The compound of any of claims 1-16 and 44-81, wherein R₄ is selected from the group of C₁-C₃ haloalkyl and halogen; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

86. The compound of any of claims 1-16 and 44-81, wherein R₄ is selected from the group of C₁-C₃ fluoroalkyl and F; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

87. The compound of any of claims 1-16 and 44-81, wherein R₄ is selected from the group of —NH—S(═O)₂H and —NH—S(═O)₂—C₁-C₃ alkyl; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

88. The compound of any of claims 1-16 and 44-81, wherein R₄ is H; or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

89. A compound selected from the group of: 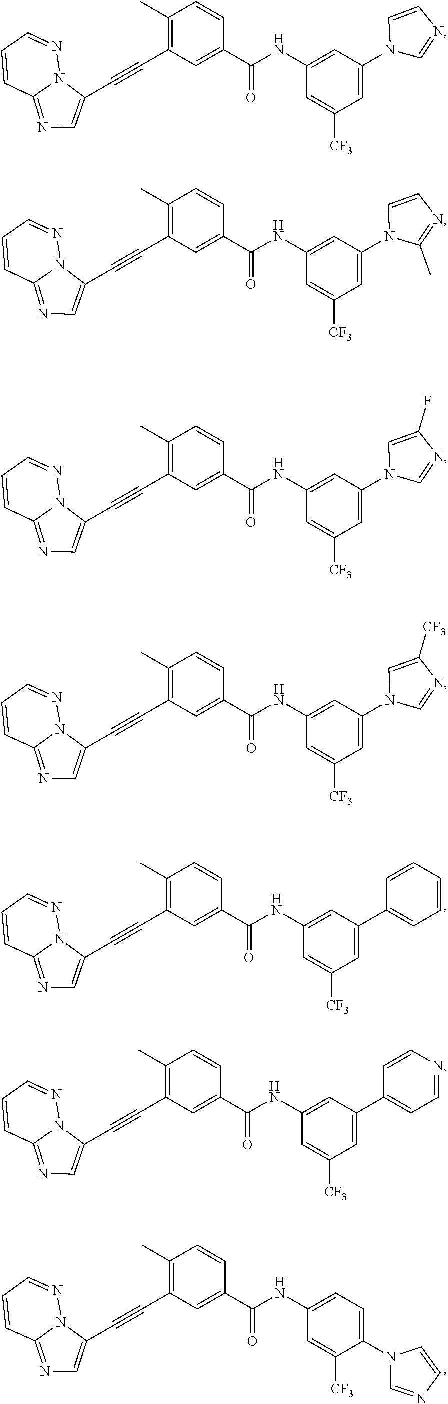 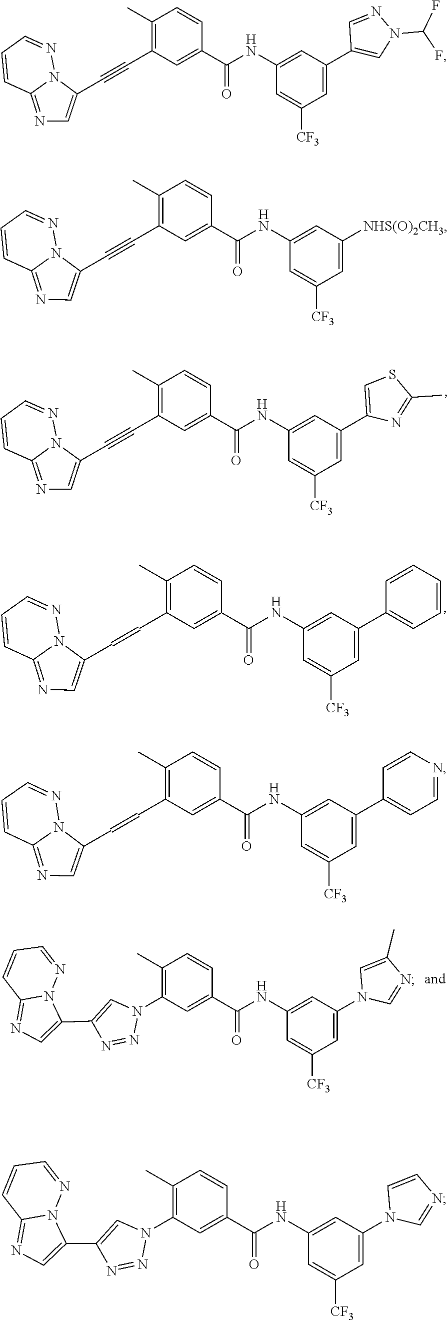
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, and a pharmaceutically acceptable carrier or excipient.

90. A pharmaceutical composition comprising a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, and a pharmaceutically acceptable carrier or excipient.

91. The use of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in the preparation of a medicament.

92. A method of inhibiting the proliferation of cancer cells in a subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

93. A method of inhibiting the proliferation of BCR-ABL-expressing cells in a subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

94. A method of treatment of chronic myeloid leukemia (CML) in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

95. A method of treatment of chronic phase chronic myeloid leukemia (CML) in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

96. A method of treatment of accelerated phase chronic myeloid leukemia in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

97. A method of treatment of blast phase chronic myeloid leukemia (CML) in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof

98. A method of treatment of acute lymphoblastic leukemia (ALL) in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

99. A method of treatment of acute lymphocytic leukemia in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

100. A method of treatment of lymphoma in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

101. A method of treatment of solid tumors in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

102. A method of treatment of Ph-positive acute lymphoblastic leukemia (Ph+ ALL or Philadelphia chromosome-positive ALL) in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

103. A method of treatment of Ph-positive acute lymphoblastic leukemia (Ph+ ALL or Philadelphia chromosome-positive ALL) in a human subject, the method comprising administering to the subject in need thereof:
a) a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a therapeutically effective amount of at least one tyrosine kinase inhibitor selected from the group of imatinib (GLEEVEC®), dasatinib (SPRYCEL®), ponatinib (ICLUSIG®), bosutinib (BOSULIF®), asciminib (SCEMBLIX®), and nilotinib (TASIGNA®), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug of the one or more tyrosine kinase inhibitor(s).

104. A method of treatment of Ph-positive acute lymphoblastic leukemia (Ph+ ALL or Philadelphia chromosome-positive ALL) in a human subject, the method comprising administering to the subject in need thereof:
a) a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof; and
b) a therapeutically effective amount of blinatumomab.

105. A method of treatment of acute myelogenous leukemia (AML) in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

106. A method of treatment of a myelodysplastic syndrome in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

107. A method of treatment of gastric cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

108. A method of treatment of endometrial cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

109. A method of treatment of bladder cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

110. A method of treatment of multiple myeloma in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

111. A method of treatment of breast cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

112. A method of treatment of prostate cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

113. A method of treatment of lung cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof. A further embodiment provides such a method of treatment of lung cancer, wherein the lung cancer is non-small cell lung cancer.

114. A method of treatment of colorectal cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

115. A method of treatment of renal cancer in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

116. A method of treatment of glioblastoma in a human subject, the method comprising administering to the subject in need thereof a therapeutically effective amount of a compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

117. A method of treatment of gastrointestinal stromal tumor in a human subject, the method comprising administering to the subject in need thereof a therapeutically effective amount of a compound of Formula (I), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

118. The method of any of claims 92-117, wherein the subject in need thereof has a point mutation in the kinase domain.

119. The method of any of claims 92-118, wherein the subject in need thereof has an BCR-ABL mutation.

120. The method of any of claims 92-119, wherein the subject in need thereof has a T315I mutation.

121. The method of any of claims 92-119, wherein the subject in need thereof has a F₃₁₇L mutation.

122. The method of any of claims 92-119, wherein the subject in need thereof has a E255K mutation.

123. The method of any of claims 92-119, wherein the subject in need thereof has a Y253F mutation.

124. A compound of any of claims 1-89, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in the preparation of a medicament for use as a pan-BCR-ABL inhibitor.
